(19) United States
(12) Patent Application Publication
(10) Pub. No.: US 2010/0003269 A1
(43) Pub. Date: Jan. 7, 2010

(54) METHODS AND USES OF CAULIFLOWER AND COLLARD FOR RECOMBINANT PROTEIN PRODUCTION

(75) Inventors: Hilary Koprowski, Wynnewood, PA (US); Maxim Golovkin, Philadelphia, PA (US); Slava Andrianov, Warrington, PA (US); Sergei Spitsin, Cherry Hill, NJ (US); Natalia Pogrebnyak, Highland Park, NJ (US)

Correspondence Address: DAVID S. RESNICK, NIXON PEABODY LLP, 100 SUMMER STREET, BOSTON, MA, 02110-2131, US

(73) Assignee: THOMAS JEFFERSON UNIVERSITY, Philadelphia, PA (US)

(21) Appl. No.: 12/373,087

(22) PCT Filed: Jun. 28, 2007
(86) PCT No.: PCT/US07/15029

§ 371 Date: Jan. 9, 2009

Related U.S. Application Data
(60) Provisional application No. 60/819,996, filed on Jul. 11, 2006.
(60) Provisional application No. 60/850,890, filed on Oct. 11, 2006.

(51) Int. Cl. A61K 39/00; C12N 15/82; A01H 5/00; A61K 38/16; C07K 14/415

(52) U.S. Cl. 424/184.1; 800/288; 800/306; 514/12; 530/370

(57) ABSTRACT

The present invention relates to a method for the generation of transgenic Brassica oleracea plants, in particular transgenic Cruciferae (also known as Brassicaceae) plants. The present invention also provides a method for the production of heterologous proteins using a Cruciferae-based plant system, for example pharmaceutical and/or recombinant proteins. In particular the invention also relates to a method for the production of transgenic collard and cauliflower, and to the large scale production of pharmaceutical and/or therapeutic production, such as production of Cruciferae-based vaccine production.

### CROSS REFERENCE TO RELATED APPLICATIONS

This application claims priority under 35 U.S.C. 119(e) of U.S. Provisional Patent Application Ser. No. 60/819,996 filed 11 Jul. 2006 and Provisional Patent Application Ser. No. 60/850,890 Filed Oct. 11^{th}, 2006, the contents of which herein are incorporated by reference in their entirety.

### GOVERNMENT SUPPORT

The present application was supported by the United States Department of Agriculture (USDA) to Biotechnology Foundation Laboratories, USDA Cooperative agreement # 58-1275-303-03, and the Government of the United States has certain rights thereto.

### FIELD OF THE INVENTION

The present invention relates generally to a method for the generation of transgenic Cruciferae (also known as Brassicaceae) plants and more particularly to the large scale production of recombinant proteins from transgenic Cruciferae plants. In particular, the invention relates to a method for the production of transgenic collard and cauliflower, and to the large scale production of pharmaceutical and/or therapeutic production, such as production of Cruciferae-based vaccine production.

### BACKGROUND

Plants have emerged as a modern production system to produce recombinant proteins—antigens that can be used as pharmaceutical proteins, for example as subunit vaccines. The ideal plant candidate for this purpose should be capable to sustain high levels of expression of foreign proteins without adverse effects on its growth and development. It is also essential that it has large biomass, is edible and suitable for long-term storage and delivery.

Plant genetic transformation technology has opened a new avenue to producing complex recombinant pharmaceutical proteins [1-6]. This approach in plants has become an attractive

alternative to other technologies, since it is associated with low production cost, overall safety and scalability potential [4, 7, 8]. However, despite numerous studies, there are only a few reports of actual production of immunologically functional recombinant subunit vaccines in plants [9], and even fewer plant-derived vaccine candidates have reached clinical trials [10].

The major benefit of using plants for vaccine production is that they allow direct oral and other needle-free routes of immunization [11-12]. The plant system also avoids costly purification processes and allows simple downstream processing of transgenic material in its natural or modified forms such as powder, tablets, creams, etc [2,8]. Currently, the ideal plant for such use is considered one that is edible, suitable for long-term storage and delivery, easily grown and processed and able to sustain high levels of expression of foreign recombinant proteins without adverse effects on its growth and development [4,7].

Plant transgenic biotechnology has provided successful transformation techniques for a variety of dicot and monocot plants [13,14]. However, efforts in pharmaceutical production have been limited mainly to those model plant species that are easily transformed such as tobacco and Arabidopsis [reviewed in 3, 9]. Among the crop species used for production of recombinant vaccines are tomato [15-18], potato [19-24] and alfalfa [25-27]. Stand-alone examples also include lettuce [28] and carrot [29]. Monocot plants have also been used for production of recombinant antigens, in most cases presented in maize seeds [30] and recently reported for rice [31]. Most of these studies were done as proof-of-concept [9,10] and it has since become clear that some plants are not suitable for this purpose. In tomato, for example, antigen expression levels in the fruits can be very low and/or vary dramatically in the pool of fruits originating from the same transgenic line [15,16]. Our own recent experiments revealed severe degradation of recombinant protein in the ripened tomato fruits as compared to immature green fruits [1,8]. Potato tubers, which were once considered promising for vaccine production purposes, have shown very low levels of antigen expression and, for oral delivery, would have required heat treatment that destroys the recombinant protein [32]. In most cases reported for crops, the overall yields of recombinant proteins expression were relatively low and/or plant material was not suitable for oral administration or storage [8-10].

To address the modern requirements for production and delivery of recombinant vaccines, we are developing a Cruciferae-based system comprised of collard, cauliflower and other vegetables. Collard is a large green leafy crop that is easy to grow, survives ambient temperatures, and produces kilogram amounts of rough leaf material from a single plant. It is convenient for production of recombinant proteins in large leaf tissues using strong tissue-specific promoters and specific intracellular targeting signals. Cauliflower has an edible overgrown inflorescence (curd or head) that is also convenient for long-term storage, transportation and delivery. Like other Cruciferous vegetables, cauliflower also has proven anti-cancer bioactive sulfur-containing compounds known as glucosinolates (isothiocyanates and terpenes) [reviewed 33, 34]. Both collard and cauliflower are close relatives of Arabidopsis, a well-studied plant model system [35]. Thus, advances in Arabidopsis research, especially regarding identification of suitable mutants/knockouts and specific genetic elements, will help greatly in developing the Cruciferae-based system.

To date, production of transgenic collard plants has not been reported. Despite many attempts to transform cauliflower, only a few studies describe successful transformation events [36-39, 41]. This species is considered recalcitrant for genetic transformation due to low regeneration efficiency, high sensitivity to Agrobacterium, and difficulties with selection procedures [39-41]. None of the published reports describe the use of transgenic cauliflower for production of bio-pharmaceutical proteins.

### SUMMARY

The inventors have discovered a method for the production and generation of stable transgenic vegetable plants of the Cruciferae family, collard and cauliflower, as a part of plant-based system for production of pharmaceutical proteins. Multiple parameters were tested and optimized to achieve an efficient stable transformation of these recalcitrant species with constructs containing expression cassettes for the known viral antigens. Efficient transformation procedures were developed for these species based on the nptll and bar genes as selectable markers. Use of our original procedure led to the generation of transgenic collard that express B5 recombinant vaccine candidate against smallpox at high levels with no adverse effect on its phenotype. In the case of cauliflower, transgenic plants were obtained expressing the S1-fragment of SARS-CoV spike protein in transgenic florets.

This work is a part of an effort to develop Cruciferae-based production system using transgenic vegetable plants collard and cauliflower. Several parameters were tested and optimized to achieve an efficient stable transformation of these recalcitrant species with constructs containing expression cassettes for the known viral antigens. Using the original procedure we obtained transgenic collard cv Morris Heading that express high levels of smallpox vaccine candidate (B5) in leaves and retain its normal phenotype. Transgenic cauliflower plants cv Early Snowball were obtained in similar procedure and have shown detectable amounts of SARS coronavirus spike-protein (SARS-COV Si) in floret tissue of mature curd.

In aspect of the present invention, the methods as disclosed herein provide a method for generating transgenic Brassica oleracea, the method comprising; contacting hypocoyly or cotelydons of Brassica oleracea plant seedlings in a first medium; optionally, pre-cultivating the hypocoyly or cotelydons of Brassica oleracea plant seedlings by contacting them with a MSC callus induction media or tobacco feeder cells for at least 2 days; contacting the Brassica oleracea seedlings with a suspension of agrobacterium comprising a nucleic acid encoding a transgene of interest and a selectable marker; regenerating the Brassica oleracea in the first medium comprising an agrobacterium killing agent for a sufficient period of time, then an optional step of selecting for transgenic Brassica oleracea by contacting the Brassica oleracea with a second medium comprising a concentration of a selection agent; and a final step of inducing root growth by incubating the transgenic Brassica oleracea in a third medium comprising a higher concentration of a selection agent than the third medium and an agrobacterium killing agent, wherein incubation is for a sufficient amount of time for generation of plantlets with roots and the Brassica oleracea plants with roots are transgenic Brassica oleracea.

In some embodiments, the methods as disclosed herein can further comprise additional steps of transferring the plantlets to soil and incubating at 24° C. for at least 3 weeks followed by a step to induce seed production by incubating the plantlets to cold conditions, for example 4° C., for at least 1 month followed by a step to induce flowed generation by transferring the plantlets to 24° C. for a period of time for flower generation.

In some embodiments, the Brassica oleracea useful in the methods as disclosed herein is from the Acephala group of Brassica oleracea, for example but not limited to collard, kale and spring greens or variants or hybrids thereof. In some embodiments, where the Brassica oleracea is from the Acephala group, the hypocoyly or cotelydons at least 4 days post germination.

In other embodiments, the Brassica oleracea useful in the methods as disclosed herein is the botrytis group of Brassica oleracea, for example but not limited to cauliflower, broccoli, broccoli romanesco or broccoflower or variants or hybrids thereof. In some embodiments, where the Brassica oleracea is from the botrytis group, the hypocoyly or cotelydons at least 4 days post germination, or alternatively at least 7 days post germination.

In some embodiments, the first medium is MSO medium comprising zeatin, BAP and non-essential amino acids (NAA), zeatin and silver nitrate. In some embodiments, where the Brassica oleracea is from the Acephala group, the first medium comprises a NAA concentration is at least 0.1 mg/l. In alternative embodiments, where the Brassica oleracea is from the botrytis group, the first medium comprises a NAA concentration is at least 0.05 mg/l.

In some embodiments, the suspension of agrobacterium is at a concentration of between OD₆₀₀ of 0.1 and 0.02. For example but not limited to, where the Brassica oleracea is from the Acephala group, the concentration of suspension of agrobacterium is OD₆₀₀ of 0.1 or less than 0.1. Alternatively, in another embodiment where the Brassica oleracea is from the botrytis group, the concentration of suspension of agrobacterium is, for example, OD₆₀₀ of 0.02 or less than 0.02.

In some embodiments, an agrobacterium killing agent useful in the methods as disclosed herein is timentin.

In further embodiments, where the Brassica oleracea is from the Acephala group a sufficient amount of time is at least 8 days, or a sufficient amount of time is at least 10 days where the Brassica oleracea is from the botrytis group. In some embodiments, a selection agent depends on the selectable marker present in the agrobacteruim, for example a selectable marker gene present on a binary vector present within the agrobacteriam. Such selectable marker genes include resistant genes and the selection agents are, for example but not limited to phosphintricin (PPT) and kanomycin (Kan). In some embodiments, the concentration of the selectable agent is higher in the third medium as compared to the second medium, for example, where the concentration of PPT in the second medium is 2 mg/l and the concentration of PPT in the third medium is 3 mg/l, or for example where the concentration of Kan in the second medium is 20 mg/l and the concentration of Kan in the third medium is 30 mg/l.

In some embodiments, the Brassica oleracea plantlets with roots have roots of about 3 cm long.

Another aspect of the present invention relates to a transgenic Brassica oleracea plant produced according to the methods as disclosed herein. In some embodiments, the transgenic Brassica oleracea plant is used for the production of an immunogenic protein, for example a virus coat protein or a fragment thereof, or the production of a heterologous protein.

In another embodiment, the methods as disclosed herein are useful for producing an immunogenic protein, the method comprising producing a transgenic Brassica oleracea plant according to the methods as disclosed herein wherein the transgene is an immunogenic protein. In some embodiments, the immunogenic protein is a virus capsid, for example but not limited to a virus capsid of SARS coronavirus or a fragment or variant thereof, or a virus capsid of small pox virus or a fragment or variant thereof. In some embodiments, the immunogenic protein can be used as a vaccine, for example for to induce an immune response in a subject. In some embodiments, the immunogenic peptide can be used to generate antibodies that can be administered to a subject as a passive immunization strategy, or in alternative embodiments, the immunogenic peptide can be directly administered to a subject as a vaccine in an active immunization strategy.

A further aspect of the present invention relates to the production of a heterologous protein produced from transgenic Brassica oleracea plants according to the methods as disclosed herein. In some embodiments, the heterologous protein is a recombinant protein, for example a vaccine such as a subunit vaccine.

In such embodiments, a recombinant protein is a virus capsid of SARS coronavirus or a fragment or variant thereof. In an alternative embodiment, a recombinant protein is small pox virus or a fragment or variant thereof.

Another aspect of the present invention provides a method for vaccinating a subject for a disease, the method comprising administering an effective amount of an immunogenic protein produced according to the methods as disclosed herein, wherein an immune response to the immunogenic protein in a subject is effective at reducing at least a symptom of the disease. In some embodiments, administration is by eating a part of the transgenic Brassica oleracea plant, for example but not limited to at least one flower and/or at least one leaf.

### BRIEF DESCRIPTION OF FIGURES

FIG. 1 shows the assessment of regeneration and transformation procedures for collard. Panel 1A shows the Regeneration response for cv Morris Heading after 3 weeks on MSR-1 medium: hypocotyl explants demonstrate shoot regeneration as well as callus formation (left); strong shoot regeneration was obtained from cotyledon explants (right). Panel 1B shows Transgenic shoot formation (left) and development of morphologically normal transgenic plant (right) in the presence of the selection agent PPT. Panel 1C shows Root induction conducted on PPT selective medium was visibly suppressed and non-transgenic shoots were eventually bleached (left) as compared with the normal growth of transgenic lines developing roots (right).

FIG. 2 shows the evaluation of parameters critical for efficient collard transformation. Panel 2 A shows the shoot regeneration efficiency in three cultivars Georgia, Yates and Morris Heading. Cv Morris Heading showed a slightly better shoot regeneration capacity from hypocotyls and cotyledons as compared to the other cultivars. Regeneration frequency is expressed as the percent of explants producing shoots on MSR-1 medium during 5 weeks. Panel 2 B shows the effects of Agrobacterium concentration and co-cultivation time on transformation efficiency of cv Morris Heading. Explants were dipped in inoculums at three different concentrations (OD₆₀₀-0.5, -0.3 and -0.1) and co-cultivated for 2 or 3 days. Transformation frequency was calculated as the percent of explants producing transgenic lines. Panel 2 C shows the respective effects of pre-cultivation and feeder layer application on transformation efficiency. Note the decrease in transformation efficiency from the initial 12% (dashed line) as compared to the 2- and 4-day pre-cultivation periods and application of a feeder layer. Panel 2D shows the effect of delay period on transformation efficiency. After co-cultivation explants were placed on medium without selection for 4, 8 or 12 days and then transferred for selection. The highest efficiency was obtained using the 8-day delay period.

FIG. 3 shows the generation of transgenic collard lines expressing recombinant viral antigen against smallpox in leaf tissue. Panel 3A shows fully developed transgenic plants were grown on PPT selection medium (left) and transferred to soil to mature (right). Panel 3b shows PCR analysis confirmed the presence of recombinant DNA in selected transgenic plants (lines 1-4) and not in the non-transgenic wild-type (wt) plant. PCR product of the same molecular weight was detected in the positive DNA control (+), as indicated by the arrowhead. Panel 3C shows three-month-old transgenic plants (B5+) were grown in standard greenhouse conditions (example shown in the right panel) and revealed no morphological differences from wild-type (wt) plant of the same age (left). Panel 3D shows western blot analysis confirmed the presence of expected molecular size foreign protein B5 (37 kDa) in a transgenic line (indicated by arrowhead) as detected by 206C5-F12-protein-specific monoclonal antibodies (mAb). Panel 3E shows a flowering transgenic collard plant that was induced by prolonged cold treatment (>1 month at 4° C.).

FIG. 4 shows the generation of transgenic cauliflower plants expressing recombinant SARS-CoV S1 antigen in mature curd flower head. Panel 4A shows transgenic cauliflower plants were grown under Km selection (Km^{R}) for 1.5 months (left) and then transferred into soil (right) to mature. Panel 4B shows PCR analysis confirming the presence of the transgene in plant DNA. Transgenic lines (i-4) and the positive DNA control (+) showed the product of expected molecular weight (arrowhead). No amplification product was detected in the genomic DNA of non-transgenic wild-type plant (wt). Panel 4C shows an example of transgenic cauliflower plant with induced curd (left) and samples of transgenic curd florets from two different transgenic lines (right). Panel 4D shows western blot analysis of extracts from florets 1 and 2 shown in panel 4C revealed the presence of SARS CoV S1 recombinant protein of expected molecular size (79 kDa) (right panel, lanes 1 and 2) using antigen-specific monoclonal antibodies (Sn+Sm MAb). Double arrowheads indicate an additional antigen-specific band at lower molecular weight position was detected in all transgenic curd tissue samples. A single band at the correct molecular weight position (left panel) was detected in E. coli-derived positive control sample (+) and in extracts from transgenic tobacco transformed with the same construct (lanes t-1 and t-2); no product was detected in non-transgenic wild-type (wt) plants of either species.

FIG. 5 shows a schematic diagram of Cruciferae-based plant system for production of pharmaceutical proteins. Transgenic plants for three Cruciferae species (Arabidopsis, collard and cauliflower) are currently used for testing and production purposes (green circles). Transformation procedures for two other cruciferous vegetables (cabbage and broccoli) are also shown.

FIG. 6 shows a schematic diagram of the generation of transgenic Cruciferae (also known as Brassicaceae) plants for the production of recombinant proteins, for example for production of immunogenic proteins and assessment of an induction of an immune response when administered to a subject.

FIG. 7 shows a schematic diagram of the expression cassette for the B5 protein of the small pox virus for the production of a vaccine against smallpox.

FIG. 8 shows the assessment of regeneration and transformation procedures for collard, with the regeneration response shown, transgenic shoot formation and root induction also shown.

FIG. 9 shows the generation of stable transgenic collard expressing recombinant B5 antigen.

FIG. 10 shows the expression analysis of transgenic collard lines by western blot analysis. Transgenic lines are indicated by the arrow (<).

FIG. 11 shows a double-step purification of B5 protein for injection and intranasal immunization of a subject. The lanes correspond to the eluted sample following each step in the purification procedure, with lanes 2-5 purified using Step I procedure, and lanes 7-9 sample elutates from the purification procedure of Step II.

FIG. 12 shows the purified B5 protein for injection and intranasal immunization of mice. The purified antigen is shown in the bottom panel.

FIG. 13 shows the serum IgG levels in mice immunized with the plant-derived B5 antigen. As shown in FIGS. 13A and 13B, both parenteral and intranasal administration with plant-derived B5 antigen results in IgG levels. Panel 13b shows intranasal administration is best with CT as compared to without CT administration. Panel 13D shows a mouse eating the transgenic plant comprising the B5 antigen.

FIG. 14 shows serum IgG levels in mice immunized with the plant-derived B5 antigen administered via intranasal route in the presence of CT or CpG.

FIG. 15 shows western blot sera analysis of mice immunized with plant-derived B5 antigen as compared to bacterial derived B5 antigen. FIG. 15A shows the level of expression of B5 of serial dilutions of plant-derived or bacterial derived B5, showing a higher level of expression of plant-derived B5. Panel 15B is a western blot using anti-cmyc antibody to detect the B5 conjugated to the B5.

FIG. 16 shows serum IgG levels in pigs immunized with plant-derived B5 antigen. Administration of plant-derived B5 via intranasal or parenteral administration resulted in an immunogenic response and production of serum IgG.

FIG. 17 shows in vitro virus neutralization.

FIG. 18 shows mice challenged with live vaccinia virus (1×10⁶ pfu) and plant-derived B5. Mice administered the plant-derived B5 has a similar response as compared to mice administered the vaccine virus.

### DETAILED DESCRIPTION

The present invention relates generally to a method for the generation of transgenic Cruciferae (also known as Brassicaceae) plants and more particularly to the large scale production of recombinant proteins from transgenic Cruciferae plants. In particular, the invention relates to a method for the production of transgenic collard and cauliflower, and to the large scale production of pharmaceutical and/or therapeutic production, such as production of Cruciferae-based vaccine production.

The inventors have discovered a method for the production and generation of stable transgenic vegetable plants of the Cruciferae family, collard and cauliflower, as a part of plant-based system for production of pharmaceutical proteins. Multiple parameters were tested and optimized to achieve an efficient stable transformation of these recalcitrant species with constructs containing expression cassettes for the known viral antigens. Efficient transformation procedures were developed for these species based on the nptll and bar genes as selectable markers. Use of our original procedure led to the generation of transgenic collard that express B5 recombinant vaccine candidate against smallpox at high levels with no adverse effect on its phenotype. In the case of cauliflower, transgenic plants were obtained expressing the S1-fragment of SARS-CoV spike protein in transgenic florets.

The inventors tested and optimized several parameters to achieve an efficient stable transformation of these recalcitrant species with constructs containing expression cassettes for the known viral antigens. Using the original procedure we obtained transgenic collard cv Morris Heading that express high levels of smallpox vaccine candidate (B5) in leaves and retain its normal phenotype. Transgenic cauliflower plants cv Early Snowball were obtained in similar procedure and have shown detectable amounts of SARS coronavirus spike-protein (SARS-COV Si) in floret tissue of mature curd.

### Definitions

The term “Cruciferae” or “Brassicaceae” are used interchangeably herein refers to the mustard family or cabbage family. The family contains species, for example cabbage, broccoli, cauliflower, brussels sprouts, collards, and kale (all cultivars of one species, Brassica oleracea), Chinese kale, rutabaga (also known as Swedish turnips or swedes), seakale, turnip, radish and kohl rabi. Other well known members of the Brassicaceae include rapeseed (canola and others), mustard, horseradish, wasabi and watercress. The term “Brassica” refers to a genus of plants in the mustard family (Brassicaceae). The members of the genus may be collectively known either as cabbages, or as mustards.

The term “Brassica oleracea” or “B. oleracea” is also referred to as “wild Cabbage” refers to a species of Brassica native to coastal southern and western Europe. Brassica oleracea is the precursor to Cabbage, and includes, for example Brussels Sprouts, Broccoli, Kohlrabi, Cauliflower, Kale, and Brocciflower (a hybrid of Broccoli and Cauliflower). Brassica oleracea has been bred into a wide range of cultivars, for example, cabbage, broccoli, cauliflower, and others. The cultivars of B. oleracea are grouped by developmental form into seven major cultivar groups, Brassica oleracea Acephala Group (for example kale and collard greens (borekale)); Brassica oleracea Alboglabra Group (for example kai-lan (Chinese broccoli)); Brassica oleracea Botrytis Group (for example cauliflower (and Chou Romanesco)); Brassica oleracea Capitata Group (for example cabbage); Brassica oleracea Gemmifera Group (for example Brussels sprouts); Brassica oleracea Gongylodes Group (for example kohlrabi); Brassica oleracea Italica Group (for example broccoli). Some (notably Brussels sprouts and broccoli) contain high levels of sinigrin which is thought to help prevent bowel cancer. For other edible plants in the family Brassicaceae, see cruciferous vegetables.

The term “Cruciferae plants” or “Cruciferae vegetables” refers to edible plants in the family Brassicaceae. Cruciferous vegetables are one of the dominant food crops worldwide, and are considered to be healthful foods; high in vitamin C and soluble fiber and contain multiple nutrients with potent anti-cancer properties such as diindolylmethane, sulforaphane and selenium. 3,3′-Diindolylmethane in Brassica vegetables is a potent modulator of the innate immune response system with potent anti-viral, anti-bacterial and anti-cancer activity.

The term “collards” are also called “collard greens” or “borekale” refer to plants of the Brassica oleracea Acephala Group and are various loose-leafed cultivars of the cabbage plant. Collards are grown for its large, dark-colored, edible leaves and are classified in the same cultivar group as kale and spring greens to which they are extremely similar genetically.

The term “cauliflower” is a variety of the Botrytis Group of Brassica oleracea in the family Brassicaceae (the same species as broccoli). It is an annual plant that reproduces by seed. Cauliflower is extremely nutritious, and may be eaten cooked, raw or pickled. It is of the very same species as cabbage, mustard greens, and brussels sprouts, for example.

The Taxonomy of Common Cruciferous Vegetables

| | | specific | |
|---|---|---|---|
| common name | genus | epithet | variety |
| kale | Brassica | oleracea | acephala |
| collards | Brassica | oleracea | acephala |
| Chinese broccoli | Brassica | oleracea | alboglabra |
| (gai laan) | | | |
| cabbage | Brassica | oleracea | capitata |
| brussel sprout | Brassica | oleracea | gemmifera |
| kohlrabi | Brassica | oleracea | gongylodes |
| broccoli | Brassica | oleracea | italica |
| broccoflower | Brassica | oleracea | italica × botrytis |
| broccoli romanesco | Brassica | oleracea | botrytis/italica |
| cauliflower | Brassica | oleracea | botrytis |
| wild broccoli | Brassica | oleracea | oleracea |
| bok choy | Brassica | rapa | chinensis |
| mizuna | Brassica | rapa | nipposinica |
| broccoli rabe | Brassica | rapa | parachinensis |
| flowering cabbage | Brassica | rapa | parachinensis |
| chinese cabbage, | Brassica | rapa | pekinensis |
| napa cabbage | | | |
| turnip root; greens | Brassica | rapa | rapifera |
| rutabaga | Brassica | napus | napobrassica |
| siberian kale | Brassica | napus | pabularia |
| canola/rape seeds; | Brassica | napus | oleifera |
| greens | | | |
| wrapped heart mustard | Brassica | juncea | rugosa |
| cabbage | | | |
| mustard seeds, brown; | Brassica | juncea | |
| greens | | | |
| mustard seeds, white | Brassica | hirta | |
| mustard seeds, black | Brassica | nigra | |
| tatsoi | Brassica | rosularis | |
| ethiopian mustard | Brassica | carinata | |
| radish | Raphanus | sativus | |
| daikon | Raphanus | sativus | longipinnatus |
| horseradish | Armoracia | rusticana | |
| Japanese horseradish | Wasabia | japonica | |
| (wasabi) | | | |
| arugula | Eruca | vesicaria | |
| watercress | Nasturtium | officinale | |
| cress | Lepidium | sativum | |

In some embodiment, the immunogenic peptide is a coat protein or capsid of a virus. Non-limiting examples of viral infections are as follows; Respiratory viral infections are, for example, common cold (caused by Picornaviruses [e.g. rhinoviruses], Influenza viruses or respiratory syncytial viruses), Influenza (caused by influenza A or influenza B virus), Herpesvirus Infections (herpes simplex, herpes zoster, Epstein-Barr virus, cytomegalovirus, herpesvirus 6, human herpesvirus 7, or herpesvirus 8 (cause of Kaposi's sarcoma in people with AIDS), central nervous system viral infections (e.g. Rabies, Creutzfeldt-Jakob disease (subacute spongiform encephalopathy), progressive multifocal leukoencephalopathy (rare manifestation of polyomavirus infection of the brain caused by the JC virus), Tropical spastic paraparesis (HTLV-I), Arbovirus infections (e.g. Arbovirus encephalitis, yellow fever, or dengue fever), Arenavirus Infections (e.g Lymphocytic choriomeningitis), hemorrhagic fevers (e.g. Bolivian and Argentinean hemorrhagic fever and Lassa fever, Hantavirus infection, Ebola and Marburg viruses). One example of a common virus is Human immunodeficiency virus (HIV) infection is an infection caused by HIV-1 or HIV-II virus, which results in progressive destruction of lymphocytes. This leads to acquired immunodefciency syndrome (AIDS). Other viruses include for example Hepatitis A, hepatitis B, hepatitis C, SARS, avian flu etc.

The present invention also relates to a pharmaceutical composition comprising a heterologous protein produced from transgenic Bassica oleracea plants as disclosed herein in a pharmaceutically acceptable carrier. Effective doses of the pharmaceutical composition comprising a heterologous protein produced from transgenic Bassica oleracea plants can be delivered prophylactic or therapeutic treatment, for example for administration to induce an immune response, for example when the heterologous protein produced from transgenic Bassica oleracea plants is an immunogenic protein. In some embodiments, the immunogenic peptide can be used as a vaccine, for example for a therapy or as a prophylactic treatment.

Accordingly, the dosage and frequency of administration can vary depending on whether the treatment is prophylactic or therapeutic. In prophylactic applications, a relatively low dosage is administered at relatively infrequent intervals over a long period of time. Some subjects continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

In some embodiments, the subject is a human, and in alternative embodiments the subject is a non-human mammal. Treatment dosages need to be titrated to optimize safety and efficacy. The amount of immunogenic peptide depends on the immunogenic peptide being administered as well as whether another antigen, for example an adjuvant is also administered, with higher dosages being required in the absence of adjuvant. The amount of an immunogenic peptide for administration sometimes varies from 1 μg-500 μg per subject and more usually from 5-500 μg per administration for human administration. Occasionally, a higher dose of 0.5-5 mg per injection is used. Typically about 10, 20, 50 or 100 μg heterologous protein produced from transgenic Bassica oleracea plants is used for administration to a human.

The timing of administration can vary significantly from once a day, to once a year, to once a decade. Generally, in accordance with the teachings provided herein, effective dosages can be monitored by obtaining a fluid sample from the subject, generally a blood serum sample, and determining the titer of antibody developed against the immunogenic peptide, using methods well known in the art and readily adaptable to the specific antigen to be measured. Ideally, a sample is taken prior to initial dosing; subsequent samples are taken and titered after each immunization. Generally, a dose or dosing schedule which provides a detectable titer at least four times greater than control or “background” levels at a serum dilution of 1:100 is desirable, where background is defined relative to a control serum or relative to a plate background in ELISA assays. Titers of at least 1:1000 or 1:5000 are preferred in accordance with the present invention.

On any given day that a dosage of heterologous protein, such as an immunogenic protein produced from transgenic Bassica oleracea plants is given, the dosage is greater than about 1 μg/subject and usually greater than 10 μg/subject if adjuvant is also administered, and greater than 10 μg/subject and usually greater than 100 μg/subject in the absence of adjuvant. Doses for individual immunogenic protein selected in accordance with the present invention, are determined according to standard dosing and titering methods, taken in conjunction with the teachings provided herein. A typical regimen consists of an immunization followed by booster injections at time intervals, such as 6 week intervals. Another regimen consists of an immunization followed by booster injections 1, 2 and 12 months later. Another regimen entails an injection every two months for life. Alternatively, booster injections can be on an irregular basis as indicated by monitoring of immune response. A typical regimen consists of an immunization followed by booster injections at 6 weekly intervals. Another regimen consists of an immunization followed by booster injections 1, 2 and 12 months later. Another regimen entails an injection every two months for life. Alternatively, booster injections can be on an irregular basis as indicated by monitoring of immune response. For passive immunization with an antibody, for example against the peptide immunogen the dosage ranges from about 0.0001 to 100 mg/kg, and more usually 0.01 to 5 mg/kg of the host body weight.

For passive immunization with an immunogenic protein produced from transgenic Bassica oleracea plants, the dosage ranges from about 0.0001 to 100 mg/kg, and more usually 0.01 to 5 mg/kg, of the host body weight. For example dosages can be 1 mg/kg body weight or 10 mg/kg body weight. An exemplary treatment regime entails administration once per every two weeks or once a month or once every 3 to 6 months. In some methods, two or more monoclonal antibodies with different binding specificities are administered simultaneously, in which case the dosage of each antibody administered falls within the ranges indicated. An immunogenic protein produced from transgenic Bassica oleracea plants can be administered on multiple occasions. Intervals between single dosages can be weekly, monthly or yearly. Intervals can also be irregular as indicated by measuring blood levels of the titer of antibody developed against the peptide immunogen. Alternatively, an immunogenic protein produced from transgenic Bassica oleracea plants can be administered as a sustained release formulation, in which case less frequent administration is required. In embodiments immunogenic protein produced from transgenic Bassica oleracea plants, the dosage and frequency vary depending on the half-life of the immunogenic protein in the subject.

The pharmaceutical compositions comprising an immunogenic protein produced from transgenic Bassica oleracea plants as disclosed herein for inducing an immune response can be administered by parenteral, topical, intravenous, oral, subcutaneous, intraperitoneal, intranasal or intramuscular means for prophylactic and/or therapeutic treatment. Typical routes of administration of an immunogenic peptide are intramuscular (i.m.), intravenous (i.v.) or subcutaneous (s.c.), although other routes can be equally effective. Intramuscular injection is most typically performed in the arm or leg muscles. In some methods, the immunogenic protein produced from transgenic Bassica oleracea plants as disclosed herein or other pharmaceutical compositions are injected directly into a particular tissue, for example a tumor tissue where the immunoglobulin producing cell is located. Such administration is termed intratumoral administration. In some methods, particular pharmaceutical compositions comprising the immunogenic protein produced from transgenic Bassica oleracea plants for the treatment of diseases of the brain are administered directly to the head or brain via injection directly into the cranium. In some methods, immunogenic protein produced from transgenic Bassica oleracea plants can be administered as a sustained release composition or device, such as a Medipad™ device.

Immunogenic protein produced from transgenic Bassica oleracea plants as disclosed herein can optionally be administered in combination with other agents that are at least partly effective in treatment of other diseases. The immunogenic protein produced from transgenic Bassica oleracea plants can also be administered in conjunction with other agents used in the treatment of viral diseases and infections, for example anti-viral therapies, reverse transcription inhibitors, protease inhibitors and the like.

In some embodiments, immunogenic protein produced from transgenic Bassica oleracea plants can be optionally administered in combination with an adjuvant. A variety of adjuvants can be used in combination with an immunogenic protein produced from transgenic Bassica oleracea plants to elicit an immune response. In some embodiments the adjuvants augment the intrinsic response to the immunogenic protein produced from transgenic Bassica oleracea plants without causing conformational changes in the immunogen that affect the qualitative form of the response. In some embodiments the adjuvants is Freud's Complete Adjuvant. In alternative embodiments, the adjuvant is, for example but not limited to alum, 3 De-O-acylated monophosphoryl lipid A (MPL™) (see GB 2220211). QS21 is a triterpene glycoside or saponin isolated from the bark of the Quillaja Saponaria Molina tree found in South America (see Kensil et al., in Vaccine Design: The Subunit and Ajuvant Approach (eds. Powell & Newman, Plenum Press, N.Y., 1995); U.S. Pat. No. 5,057,540). Other adjuvants useful are oil in water emulsions (such as squalene or peanut oil), optionally in combination with immune stimulants, such as monophosphoryl lipid A (see Stoute et al., N. Engl. J. Med. 336, 86-91 (1997)). Another adjuvant is CpG (Bioworld Today, Nov. 15, 1998). Alternatively, the immunogenic protein produced from transgenic Bassica oleracea plants can be coupled to an adjuvant. For example, a lipopeptide version of an immunogenic peptide can be prepared by coupling palmitic acid or other lipids directly to the N-terminus of the immunogenic peptide as described for hepatitis B antigen vaccination (Livingston, J. Immunol. 159, 1383-1392 (1997)). However, such coupling should not substantially change the conformation of the immunogenic peptide as to affect the nature of the immune response thereto. Adjuvants can be administered as a component of a therapeutic composition with an active agent or can be administered separately, before, concurrently with, or after administration of the therapeutic agent.

In an alternative embodiment, the class of adjuvants is aluminum salts (alum), such as aluminum hydroxide, aluminum phosphate, aluminum sulfate. Such adjuvants can be used with or without other specific immunostimulating agents such as MPL or 3-DMP, QS-21, polymeric or monomeric amino acids such as polyglutamic acid or polylysine. Another class of adjuvants is oil-in-water emulsion formulations. Such adjuvants can be used with or without other specific immunostimulating agents such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1-2dipalmitoyl-sn-glycero-3-hydroxyphos phoryloxy)-ethylamine (MTP-PE), N-acetylglucsaminyl-N-acetylmuramyl-L-Al-D-isoglu-L-Ala-dipalmitoxy propylamide (DTP-DPP) theramide™, or other bacterial cell wall components. Oil-in-water emulsions include (a) MF59 (WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton Mass.), (b) SAF, containing 10% Squalene, 0.4% Tween 80, 5% pluronic-blocked polymer L121, and thr-MDP, either microfluidized into a submicron emulsion or vortexed to generate a larger particle size emulsion, and (c) Ribi™ adjuvant system (RAS), (Ribi Immunochem, Hamilton, Mont.) containing 2% squalene, 0.2% Tween 80, and one or more bacterial cell wall components from the group consisting of monophosphoryl lipid A, trehalose dimycolate (TDM), and cell wall skeleton (CWS), preferably MPL+CWS (DetoX™). Another class of preferred adjuvants is saponin adjuvants, such as Stimulon™ (QS-21; Aquila, Framingham, Mass.) or particles generated therefrom such as ISCOMs (immunostimulating complexes) and ISCOMATRIX. Other adjuvants include Incomplete Freund's Adjuvant (IFA), cytokines, such as interleukins (IL-1, IL-2, and IL-12), macrophage colony stimulating factor (M-CSF), and tumor necrosis factor (TNF). Such adjuvants are generally available from commercial sources.

An adjuvant can be administered with an immunogenic protein produced from transgenic Bassica oleracea plants as a single composition, or can be administered before, concurrent with or after administration of the immunogenic protein produced from transgenic Bassica oleracea plants. Immunogenic protein produced from transgenic Bassica oleracea plants and adjuvants can be packaged and supplied in the same vial or can be packaged in separate vials and mixed before use. Immunogenic protein produced from transgenic Bassica oleracea plants and adjuvants are typically packaged with a label indicating the intended therapeutic application. If the immunogenic protein produced from transgenic Bassica oleracea plants and adjuvant are packaged separately, the packaging typically includes instructions for mixing before use. The choice of an adjuvant and/or carrier depends on such factors as the stability of the formulation containing the adjuvant, the route of administration, the dosing schedule, and the efficacy of the adjuvant for the species being vaccinated. In humans, a preferred pharmaceutically acceptable adjuvant is one that has been approved for human administration by pertinent regulatory bodies. Examples of such preferred adjuvants for humans include alum, MPL and QS-21. Optionally, two or more different adjuvants can be used simultaneously. Preferred combinations include alum with MPL, alum with QS-21, MPL with QS-21, and alum, QS-21 and MPL together. Also, Incomplete Freund's adjuvant can be used (Chang et al., Advanced Drug Delivery Reviews 32, 173-186 (1998)), optionally in combination with any of alum, QS-21, and MPL and all combinations thereof.

In some embodiments, the immunogenic protein produced from transgenic Bassica oleracea plants can be administered as pharmaceutical compositions comprising an active therapeutic agent, i.e., and a variety of other pharmaceutically acceptable components. See Remington's Pharmaceutical Science (15th ed., Mack Publishing Company, Easton, Pa., 1980). The form of administration depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, non-therapeutic, non-immunogenic stabilizers and the like. However, some reagents suitable for administration to animals may not necessarily be used in compositions for human use.

Pharmaceutical compositions can also optionally comprise include large, slowly metabolized macromolecules such as proteins, polysaccharides, polylactic acids, polyglycolic acids and copolymers (such as latex functionalized sepharose, agarose, cellulose, and the like), polymeric amino acids, amino acid copolymers, and lipid aggregates (such as oil droplets or liposomes). Additionally, these carriers can function as immunostimulating agents (i.e., adjuvants), or targeting carries to target the immunogenic peptide to specific target cells or target organs, for example the bone marrow as a target organ or plasma cells as target cells.

For parenteral administration, the immunogenic protein produced from transgenic Bassica oleracea plants can be administered as injectable dosages of a solution or suspension of the substance in a physiologically acceptable diluent with a pharmaceutical carrier which can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (see Langer, Science 249, 1527 (1990) and Hanes, Advanced Drug Delivery Reviews 28, 97-119 (1997). The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications. For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins (See Glenn et al., Nature 391, 851 (1998)). Co-administration can be achieved by using the components as a mixture or as linked molecules obtained by chemical crosslinking or expression as a fusion protein. Alternatively, transdermal delivery can be achieved using a skin path or using transferosomes (Paul et al., Eur. J. Immunol. 25, 3521-24 (1995); Cevc et al., Biochem. Biophys. Acta 1368, 201-15 (1998)).

The present invention also relates to a pharmaceutical composition comprising a heterologous protein produced from transgenic Bassica oleracea plants as disclosed herein in a pharmaceutically acceptable carrier. In therapeutic applications, compositions are administered to a patient suffering from a disease, in an amount sufficient to reduce a symptom of the disease by at least 10%. An amount adequate to accomplish this is defined as a therapeutically effective dose. Amounts effective for this use will depend on the severity of the disease and the general state of the patient's health.

In most embodiments, the subject treated with pharmaceutical composition is a mammal, including humans and non-human mammals and animals in general, for example, mammals, non-human animals such as farm animals comprising, but not limited to: cattle, horses; goats; sheep; pigs; donkeys; etc. household pets including, but not limited to: cats; dogs; rodents comprising but not limited to: rabbits, mice; hamsters; etc; birds and poultry and other livestock and fowl.

Advantageously, the pharmaceutical composition is suitable for parenteral administration. The pharmaceutical composition comprising a heterologous protein produced from transgenic Bassica oleracea plants as disclosed herein can be administered by various means appropriate for different purposes, for example, for treating tumors in various parts of the body, according to methods known in the art for other similar compositions, such as immunotoxins (See, for example, Rybak, et al., Human Cancer Immunology, in IMMUNOLOGY AND ALLERGY CLINICS OF AMERICA, W. B. Saunders, 1990, and references cited therein). Accordingly, the present invention also relates to pharmaceutical compositions comprising a heterologous protein produced from transgenic Bassica oleracea plants as disclosed herein and a pharmaceutically acceptable carrier, particularly such compositions which are suitable for the above means of administration.

Single or multiple administrations of the compositions may be administered depending on the dosage and frequency as required and tolerated by the patient. In any event, the composition should provide a sufficient quantity of the proteins of this invention to effectively treat the patient.

In some embodiments, the compositions for administration comprising a heterologous protein produced from transgenic Bassica oleracea plants as disclosed herein can be preloaded onto polymetric nanoparticles and/or cataionic liposomes (Pattrick et al, 2001; Richardson et al., 2001; Sachdeva, 1998) in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of heterologous protein produced from transgenic Bassica oleracea plants in these formulations can vary widely, and will be selected primarily based on fluid volumes, viscosities, body weight and the like in accordance with the particular mode of administration selected and the patient's needs.

Thus, a typical pharmaceutical composition for intravenous administration would be about 0.01 to 100 mg per patient. Dosages from 0.1 up to about 1000 mg per patient can be used, particularly when the drug is administered to a secluded site and not into the blood stream, such as intramuscular administration. Actual methods for preparing parenterally administrable compositions will be known or apparent to those skilled in the art and are described in more detail in such publications as REMINGTON'S PHARMACEUTICAL SCIENCE, 15TH ED., Mack Publishing Co., Easton, Pa., (1980).

The pharmaceutical composition can be administered by any means known to persons skilled in the art. For example, some methods include pump, direct injection, topical application, or amelioration to a subject via intradermal, subcutaneous, intravenous, intralymphatic, intranodal, intramucosal, intranasal, or intramuscular administration.

In some embodiments, the efficacy of administering an immunogenic protein produced from transgenic Bassica oleracea plants as disclosed herein can be determined by assessing an immune response to the immunogenic protein following administration to a subject. In some embodiments, such methods entail determining a baseline value of an immune response in a subject before administering a composition comprising an immunogenic protein produced from transgenic Bassica oleracea plants, and comparing this with a value for the immune response after such treatment. A significant increase (i.e., greater than the typical margin of experimental error in repeat measurements of the same sample, expressed as one standard deviation from the mean of such measurements) in value of the immune response signals a positive treatment outcome (i.e., that administration of the composition comprising immunogenic protein produced from transgenic Bassica oleracea plants has achieved or augmented an immune response). If the value for immune response does not change significantly, or decreases, a negative treatment outcome is indicated. In general, subject undergoing an initial course of treatment with an agent are expected to show an increase in immune response with successive dosages, which eventually reaches the plateau. Administration of agent is generally continued while the immune response is increasing. Attainment of the plateau is an indicator that the administered of treatment can be discontinued or reduced in dosage or frequency.

In other methods, a control value (i.e., a mean and standard deviation) of immune response is determined for a control population. Typically the subjects in the control population have not received prior treatment. Measured values of immune response in a patient after administering a composition comprising immunogenic protein produced from transgenic Bassica oleracea plants are then compared with the control value. A significant increase relative to the control value (e.g., greater than one standard deviation from the mean) signals a positive treatment outcome. A lack of significant increase or a decrease signals a negative treatment outcome. Administration of a composition comprising immunogenic protein produced from transgenic Bassica oleracea plants is generally continued while the immune response is increasing relative to the control value. As before, attainment of a plateau relative to control values in an indicator that the administration of treatment can be discontinued or reduced in dosage or frequency.

In other methods, a control value of immune response (e.g., a mean and one standard deviation) is determined from a control population of subjects who have undergone treatment with the same immunogenic protein but it was produced from methods other than by using transgenic Bassica oleracea plants and whose immune responses have plateaued in response to treatment. Measured values of immune response in a patient are compared with the control value. If the measured level in a patient is not significantly different (e.g., more than one standard deviation) from the control value signals a positive treatment outcome.

In some embodiments, the tissue sample for analysis is typically blood, plasma, serum, urine, mucus or cerebral spinal fluid from the subject. The sample is analyzed for an immune response to any forms of the immunogenic protein that was produced from transgenic Bassica oleracea plant. The immune response can be determined from the presence of, e.g., antibodies or T-cells that specifically bind to the immunogenic peptides of the present invention. ELISA methods of detecting antibodies specific to the immunogenic peptides are commonly known in the art and are encompassed for use in the present invention. Methods of detecting reactive T-cells are known by person of ordinary skill in the art and useful in the methods of the present invention.

In some embodiments, an immunogenic protein produced from transgenic Bassica oleracea plant as disclosed herein can be administered but the methods as described above to a subject to induce an immune response. In some embodiments, one can test a subject for an immune response to the immunogenic protein as described above or by detecting IgG in the serum as disclosed in the Examples.

For further elaboration of general techniques useful in the practice of this invention, the practitioner can refer to standard textbooks and reviews in cell biology, tissue culture. General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXAMPLES

The examples presented herein relate to methods for the production of transgenic Brassica oleracea plants, for example from the Brassica oleracea plants from the Acephala group and/or the botrytis group. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### Methods

Transformation of Collard

Plant material and shoot regeneration. Seeds of collard (Brassica oleracea var acephala) cvs Morris Heading, Yates and Georgia (obtained from the Carolina seeds Co., Hartford, Conn.) were sterilized in 70% ethanol for 1 mm followed by 2% sodium hypochlorite for 20 mm. After rinsing 3 times in sterile distilled water, seeds were placed in germination medium MS-i containing MS macro- and microelements [42], 1% sucrose and 0.8% agar (see Table 1 for details on media composition). Germination and in vitro culture were carried out at 24° C. at 16 h-light/8h-dark photoperiods with light intensity of 40 j.tEIm²/s′. Cotyledons and hypocotyls of the three collard cultivars were cut from 4-, 7- and 10-day-old seedlings and placed on MSR-I regeneration medium. Ten to twelve explants per Petri dish were cultured for 5 weeks and tested for shoot regeneration efficiency.

Transformation procedures. Four-day-old cotyledon and hypocotyl explants were inoculated with Agrobacterium suspension (OD₆₀₀ of 0.5, 0.3, or 0.1) for 10 mm. After blotting dry with sterile filter paper, explants were transferred to MS-2 co-cultivation medium supplemented with acetosyringone (Table 1) and incubated in the dark for 2 or 3 days at 24° C. To determine the effect of pre-culture on transformation efficiency, explants were cultured for 2 or 4 days on MSC callus induction medium before inoculation with Agrobacterium. In another set of experiments, explants were placed on a feeder layer of tobacco cells during co-cultivation [43]. After co-cultivation explants were transferred to MS-3 regeneration medium without selection for 0-12 days and then transferred to MS-5 regeneration selection medium containing 2-mg/l phosphinotricin (PPT) (Sigma, St. Louis, Mo.). After 4-5 weeks in selection medium, regenerated green shoots (putative transformants) were formed. Healthy green shoots (1-2 cm) were excised and transferred to MS-7 selection medium supplemented with increased concentration of PPT (3 mg/l) for rooting. Selected plantlets with roots (˜3-5 cm long) were transferred to soil [Metromix, K. C. Schoefer, York, Pa.] in greenhouse conditions. Just before soil transfer, the transgenic status of the plants was confirmed by PCR. For seed production, collard plants were placed in a cold room (4° C.) for 1 month and then transferred to a plant growth chamber (24° C.). Transgenic plants were self-pollinated for production of T₁ seeds.

Optimization of Transformation Parameters for Cauliflower

Plants material and in vitro shoot regeneration: Seeds of three cauliflower (Brassica oleracea var. botrytis) cvs Early Snowball, Snowball, and All Year Around (obtained from Carolina Seeds Co., Hartford, Conn.) were sterilized and germinated as described above for collard. Cotyledons and hypocotyls were cut from 4-, 7- and 10-day-old seedlings, placed in MSR-2 regeneration medium, and evaluated 5 weeks later for regeneration efficiency.

Transformation procedure: The procedure established for transformation of collard was used for cauliflower cv Early Snowball, with some modifications. Cotyledons and hypocotyls were excised from 7-day-old seedlings, pre-cultivated for 2 days on MSC callus induction medium and inoculated with Agrobacterium suspension at several concentrations (of OD₆₀₀ of 0.1, 0.05, 0.02) for 10 mm. After blotting dry with sterile filter paper, explants were transferred to MS-2 co-cultivation medium supplemented with acetosyringone and incubated in the dark for 2 or 3 days at 24° C. Explants were then transferred to MS-4 regeneration medium without selection for 0-12 days, followed by transfer to MS-6 selection regeneration medium supplemented with 20 mg/l kanamycin (Km) [Sigma, St.]

Western blot analysis of transgenic plants. Protein extracts were prepared essentially as described [18] and resolved by 4-20% gradient SDS-polyacrylamide gel electrophoresis. After electro-blotting, viral-specific antigen was detected in transgenic collard, cauliflower and tobacco plants (as control) with B5-specific mouse antibody MAb206C5-F12 at 1/1000 dilution (obtained from Dr. S. Isaacs, Univ. of Penn, Philadelphia, Pa.) or SARS S-protein-specific Sn+Sm rabbit antibodies (cat# AP600b and cat#AP6000a) [Abgent, San Diego, Calif.] at 1/1000 dilution. Secondary detection was done using the corresponding horseradish peroxidase-conjugated anti-mouse or anti-rabbit antibodies (1/10000 dilution).

### Example 1

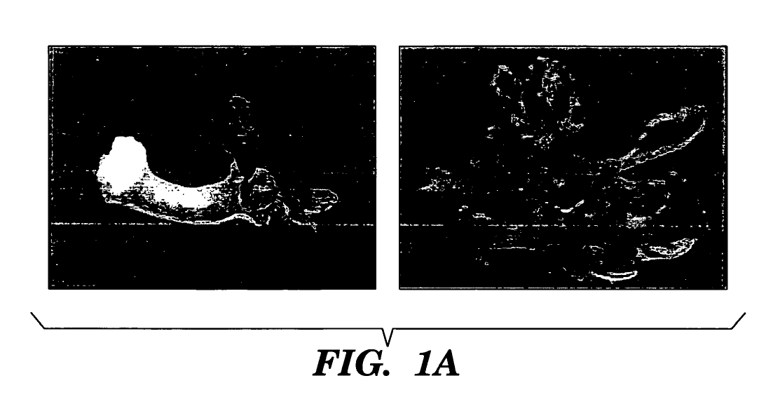

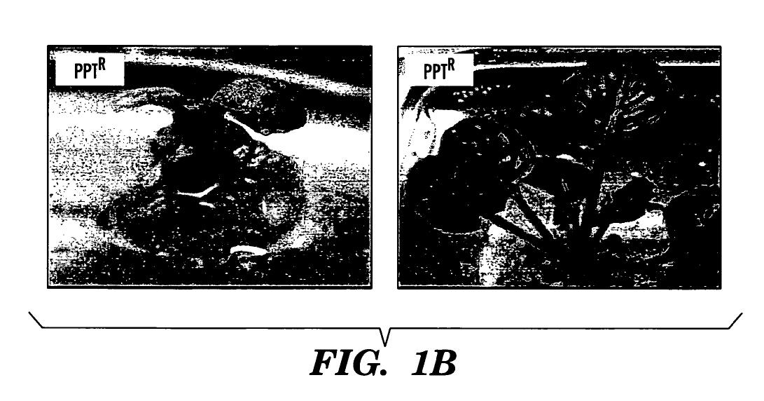

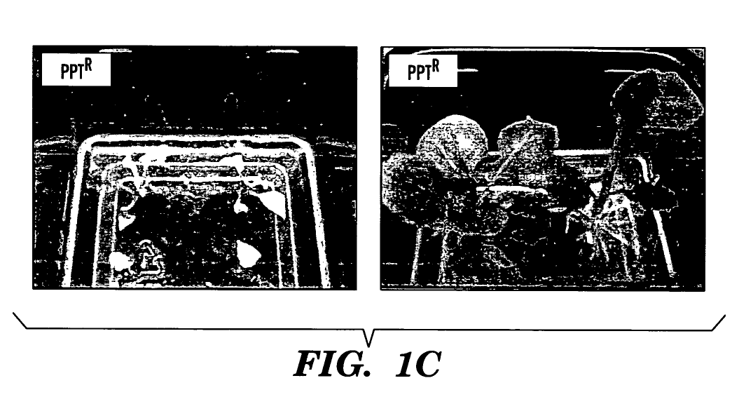

Selection of collard explant material for optimal regeneration efficiency. The inventors discovered in preliminary experiments using different media compositions and types of explants, that shoots from several commercial collard cultivars regenerated most efficiently on MS medium supplemented with the hormones zeatin, BAP, and NAA, with addition of 20 μM silver nitrate (MSR-1 medium; Table 1), and that cotyledon and hypocotyl explants had significantly better regeneration potential as compared to leaf and stem segments. The inventors discovered that during 5 weeks of growth, 70-85% of cotyledons produced shoots in MSR-1 medium and regeneration occurred directly from surface tissues, without callus formation (FIG. 1A, right).

TABLE 1
Media used for the transformation of collard and cauliflower.

| Name | Media composition |
|---|---|
| MS0 | Basic Murashige-Skoog basal medium* (MS) with 3% sucrose |
| | and 0.8% agar** |
| MSC | MS0 with 0.5-mg/l NAA, 0.5-mg/l 2.4-D, 0.5-mg/l BAP |
| MSR-1 | MSO with 20-mM AgNO3, 1-mg/l BAP, 1-mg/l zeatin, |
| | 0.1-mg/l NAA |
| MSR-2 | MSO with 20-mM AgNO3, 1-mg/l BAP, 1-mg/l zeatin, |
| | 0.05-mg/l NAA |
| MS-1 | MS medium with 1% sucrose and 0.8% agar |
| MS-2 | MSO with 100-mM acetosyringone |
| MS-3 | MSR-1 with 300-mg/l timentin |
| MS-4 | MSR-2 with 300-mg/l timentin |
| MS-5 | MS-3 with 2-mg/l phosphinotricin (PTT) |
| MS-6 | MS-4 with 20-mg/l kanamycin (Kan) |
| MS-7 | MS0 with 3-mg/l phosphinotricin (PPT) and 300-mg/l timentin |
| MS-8 | MS0 with 30-mg/l kanamycin (Kan) and 300-mg/l timentin |

*Full composition of MS medium according to original recipe [42]
**The pH of all the media used was adjusted to pH 5.7 before autoclaving

The inventors discovered hypocotyls also showed an acceptable regeneration potential (30 to 40%); however, they also tended to form callus tissues which are unable to regenerate shoots (FIG. 1A, left). Comparison of 4-, 7- and 10-day-old explants demonstrated that regeneration capacity tended to decrease sharply with age (data not shown).

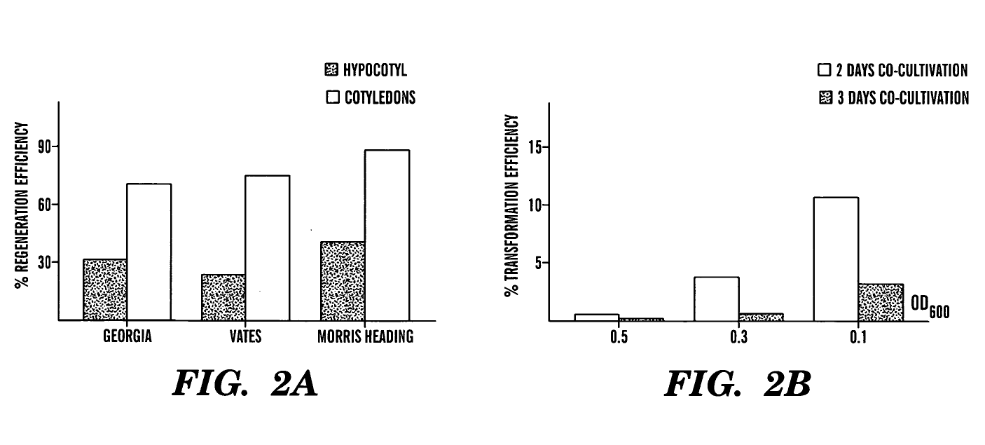

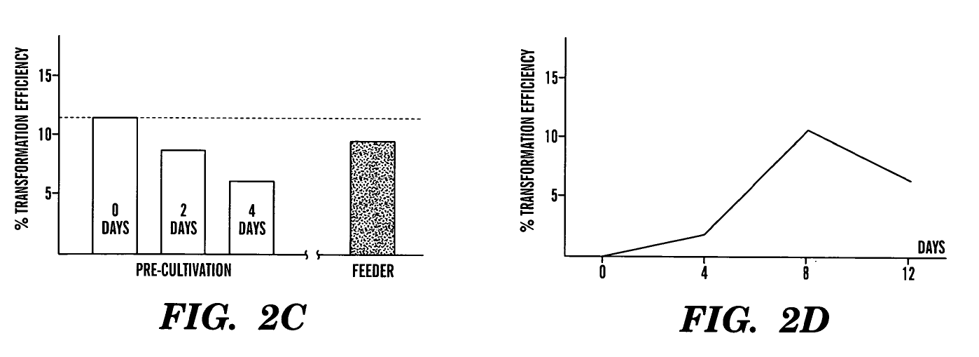

Comparison of regeneration capacity of three commercial collard cultivars (Georgia, Yates and Morris Heading revealed the highest regeneration efficiency in Morris Heading cotyledons and hypocotyls, reaching 85% and 40%, respectively (FIG. 2A). Based on these data, the inventors selected 4-day-old cotyledons and hypocotyls of cv Morris Heading for subsequent transformation experiments.

Agrobacterium-mediated transformation of collard. Preliminary transformation experiments revealed several problems associated with inoculation, co-cultivation and selection procedures. One of the most important was the necrosis of collard tissues after exposure to Agrobacterium, leading to low transformation efficiency. Indeed, the exposure of explants to overnight Agrobacterium culture at OD₆₀₀ 0.5 caused severe necrosis in most of the treated collard explants and, in turn, a transformation efficiency of less than 1%. However, the inventors discovered that explants inoculated with a suspension of Agrobacterium culture diluted to OD₆₀₀ 0.1 increased the overall transformation efficiency to 4-11% (FIG. 2B) and less necrosis. In the same set of experiments, the inventors also showed that 2 days of co-cultivation led to higher transformation efficiency than after 3 days (FIG. 2B).

In an effort to further reduce necrosis of explants in response to agrobacteria and thus improve overall transformation efficiency, the inventors did the following: i) pre-cultivating of cotyledon and hypocotyl explants in MSC induction medium for 2 or 4 days before inoculation with agrobacteria; and ii) using a feeder layer of tobacco cells during co-cultivation. Both were discovered to be unsuccessful and actually led to a decrease in transformation efficiency (FIG. 2C).

No transgenic plants were recovered when selection was started immediately after the co-cultivation. Therefore, the inventors tested the effect of delay period, during which infected explants were kept in MS-3 non-selective medium supplemented with timentin for Agrobacterium elimination for 4, 8, or 12 days. The inventors discovered transgenic shoot regeneration from explants was highest (12%) when infected explants were left on MS-3 medium for 8 days before selection (FIG. 2D), whereas while a prolonged delay period (12 days) led to a higher percentage of regenerants it also produced a lower transformation efficiency (6%) due to the larger number of escapes.

The inventors selected collard transgenic plants by growing them on medium supplemented with the selective agent PPT (FIG. 1B). The inventors discovered use of low concentrations of PPT (2 mg/l) for first selection (medium MS-5) revealed the survival of a high percentage of non-transgenic shoots. The inventors then performed a second round of selection on selection medium MS-7 in the presence of increased concentration of PPT (3 mg/l), which resulted in root induction. Under these conditions, transgenic shoots developed roots (FIG. 1C, right panel), whereas non-transgenic collard shoots were unable to form roots on this selection medium and eventually died (FIG. 1C, left panel).

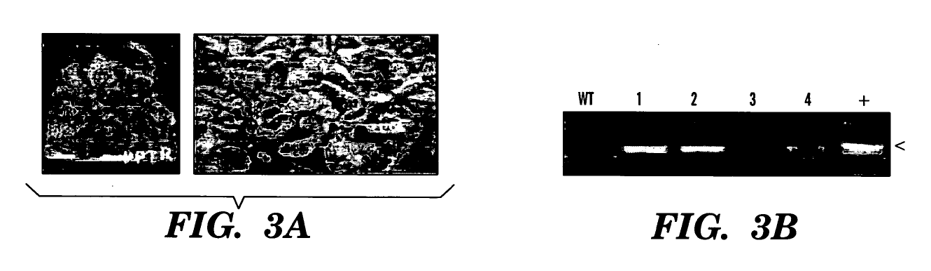

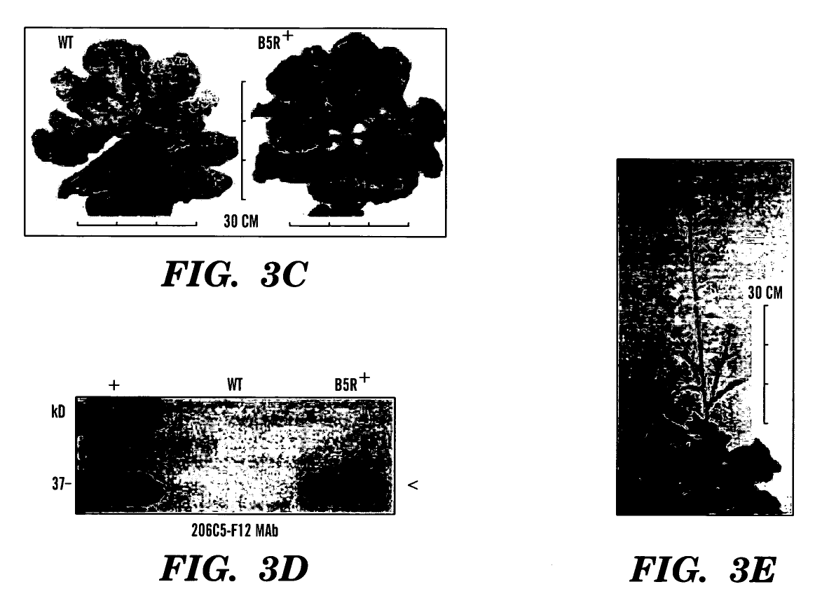

Green plants with developed roots were confirmed by PCR and transferred to soil and grown to maturity under greenhouse conditions (FIG. 3A). Transgenic 3-4 month-old Morris Heading plants were moved from the greenhouse (24° C.) to cold conditions (4° C.) for 1 month and then returned to 24° C. Three-four weeks-after cold treatment, collard plants started to flower (FIG. 3E).

Together, the inventors have discovered and demonstrated a simple and efficient protocol for collard cv Morris Heading transformation with a final efficiency of 11%. Transgenic plants grew to maturity and produced T₁ seeds.

### Example 2

Optimization of transformation parameters for cauliflower. The inventors discovered that transformation procedure developed for collard was also suitable for production of transgenic cauliflower. Three commercial cauliflower cultivars were tested in experiments for regeneration potential similar to those described for collard. Explants from cv Early Snowball showed the best regeneration potential. The inventors show that only minor adjustments made to the original procedure allowed generation of transgenic cauliflower plants.

The inventors discovered that explants from cauliflower showed better regeneration capacity (˜80%) when excised from 4-day-old seedlings and placed on MSR-2 medium with decreased concentrations of NAA compared to those in MSR-1 (see Table 1). However, cauliflower explants at this age were still tiny, fragile and extremely sensitive to Agrobacterium exposure, developing a strong necrosis response. Therefore the inventors used explants excised from 7-day-old seedlings. These were more robust and still retained a good regeneration capacity. The inventors also used a lower concentration of the Agrobacterium inoculum of OD₆₀₀ 0.02. The inventors discovered a 2-day pre-cultivation step was found to be important for cauliflower and an extended delay period of at least 10 days (as compared to 8 days for collard) was required for development of regenerable compact green tissues. With these few changes, the overall explant survivability and subsequent transformation/regeneration efficiency was increased for this recalcitrant species.

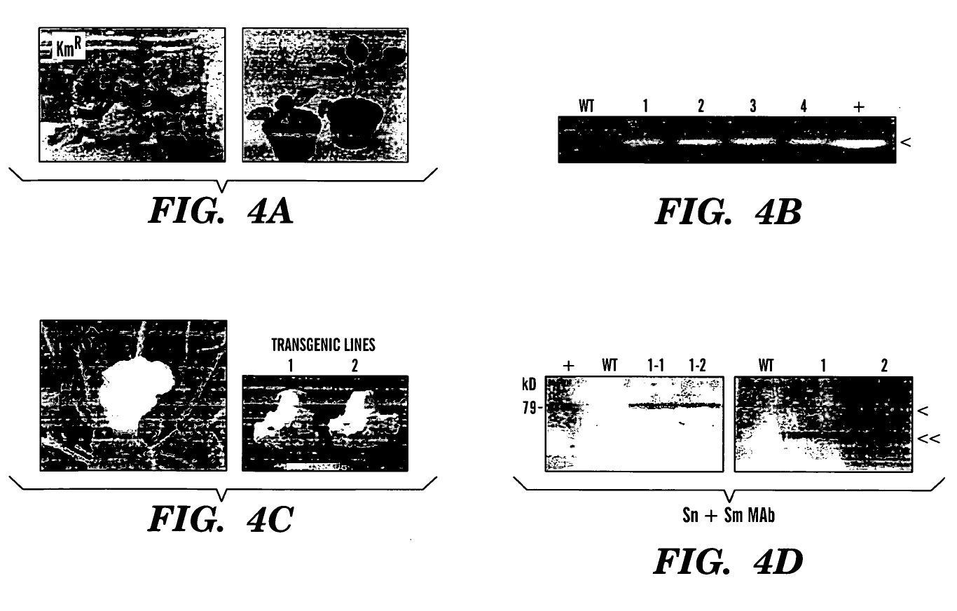

Kanamycin was used as the selection agent for transgenic cauliflower plants. The Km concentration for primary shoot formation was determined to be 20 mg/l, where some of the non-transgenic shoots were still able to survive. Thus, for root induction selection medium, the Km concentration of Kan was increased to 30 mg/l. Transgenic plants that developed roots under these conditions were confirmed by PCR and transferred to soil until development of a head (FIG. 4A). Together the inventors have discovered and demonstrated that the procedure for production of transgenic collard plants can be adapted for the production of transgenic cauliflower plants with a few modifications. The inventors have demonstrated production of transgenic cauliflower cv Early Snowball with transformation efficiency 2.4%.

### Example 3

### Production of Recombinant Subunit Vaccines in Transgenic Plants

Transgenic collard plants expressing viral coat protein B5 (smallpox antigen). The inventors show constitutive expression of Smallpox antigen in transgenic collard by stable Agrobacterium-mediated transformation of cv Morris Heading with a binary vector carrying an expression cassette with B5 gene driven by CaMV-35S promoter (see Materials and Methods). Almost all putative transgenic lines that produced roots on MS-7 medium containing phosphinotricin (PPT) (FIG. 3A) were confirmed by PCR for the presence of antigen-specific DNA (examples are shown in FIG. 3B, lanes 1-4), whereas non-transgenic wild-type (wt) collard revealed none. Leaf tissues of transgenic lines were tested for level of expression of Smallpox antigen. Western blot analysis with antigen-specific antibodies revealed a single protein band of the expected molecular size in the leaf tissue of transgenic plants (shown for the best expressing line in FIGS. 3C and D). These plants had no visible morphological changes as compared to a non-transgenic wild-type plant of the same age (FIG. 3C). Once in soil, plants produced large green leafy biomass amounts with the total weight of fresh tissue of more than 1 kg, and after 3 months reached a height of 50 cm and rosette diameter of 60 cm. Upper, medium and lower leaves were tested and confirmed for the presence of the antigen (data not shown).

### Example 4

Transgenic cauliflower plants expressing viral spike protein (SARS antigen). The inventors show expression of the 79-kD fragment of the SARS-CoV spike protein in transgenic cauliflower using a binary vector with nptll gene for selection of transgenic plants on kanamycin-containing medium [18]. Km-resistant putative transgenic shoots were generated within 5 to 6 weeks in MS-6 medium and placed in the MS-8 selection medium with increased Km content for root induction (FIG. 4A). Rooted Km^{R} (kanomycin resistant) plants tested by PCR analysis revealed the antigen-specific product of correct 338-bp size in genomic DNA of transgenic plants and control plasmid DNA (FIG. 4B, lanes 1-4 and /+/lane, respectively). This product was not present in the DNA sample of non-transformed wild-type plants (FIG. 4B). PCR-positive transgenic plants were placed in soil to grow and form heads. Western blot analysis using SARS antigen-specific Sn+Sm antibodies [18] revealed the antigen-specific band of expected molecular weight in florets of several cauliflower transgenic lines and not in the wild-type floret sample (FIG. 4D, right panel, arrowhead). A second specific band of lower molecular weight was present in all transgenic floret samples (FIG. 4D, right panel, double-arrowhead). In control experiments, only one band was detected in the leaf tissue of transgenic tobacco plants transformed with the same construct (FIG. 4D, left panel, lanes 1 and 2). A protein product of almost the same size was detected in E. coli extracts with induction of the same expression cassette (FIG. 4D, left panel, lanes +). Some transgenic cauliflower plants showed slight inhibition of growth as compared to non-transformed plants. SARS-CoV S1 antigen was easily detected in transgenic floret samples stored for as long as 5 months at −80° C.

Herein, the inventors have clearly demonstrated the feasibility of recombinant protein expression in cruciferous plants. The inventors have overcome the use of production of recombinant protein expression in plants using commonly used viral mechanism. Also, the inventors have overcome the toxicity-associated problems with production of viral-mediated recombinant protein expression in plants, such as occurring in the over-expression of large full-size viral antigens, such as rabies G-protein or SARS spike protein which perturbs the normal growth and development of the plants, which is most likely due to the induction of natural plant defense mechanism against some viral pathogens [46-48].

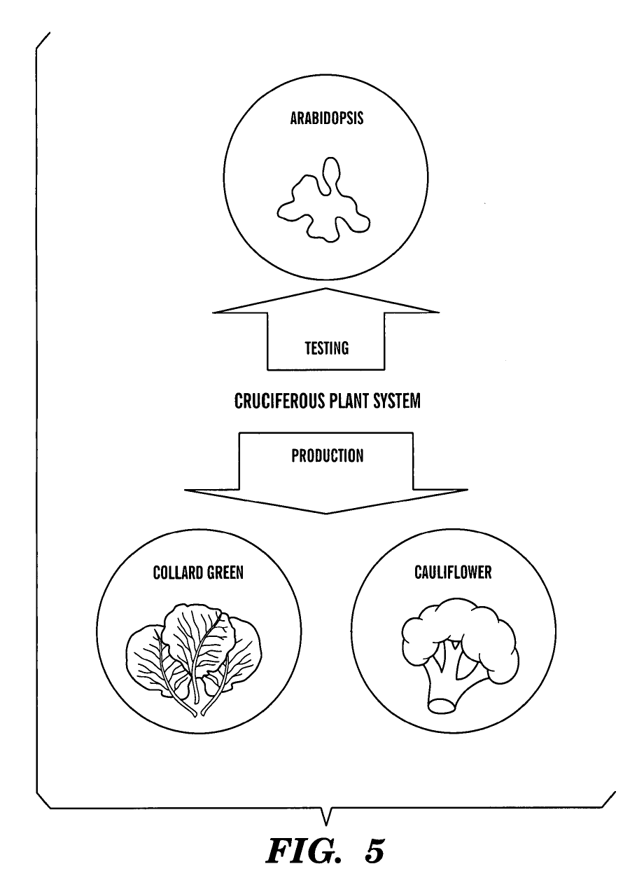

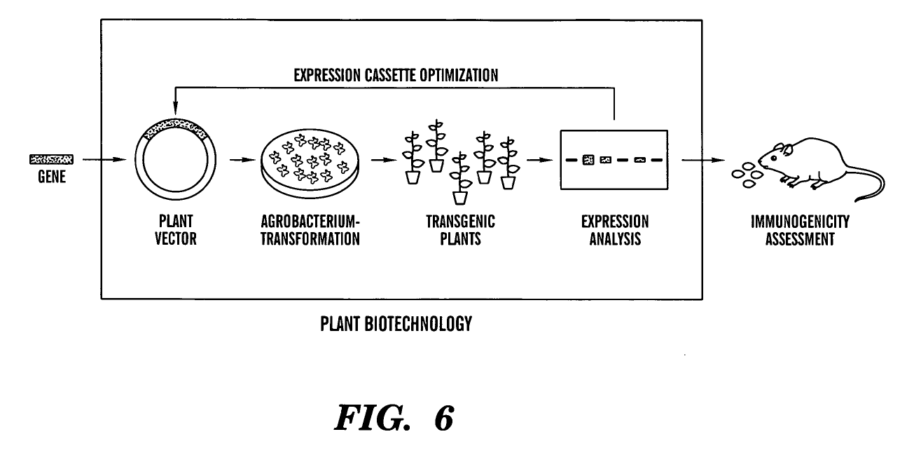

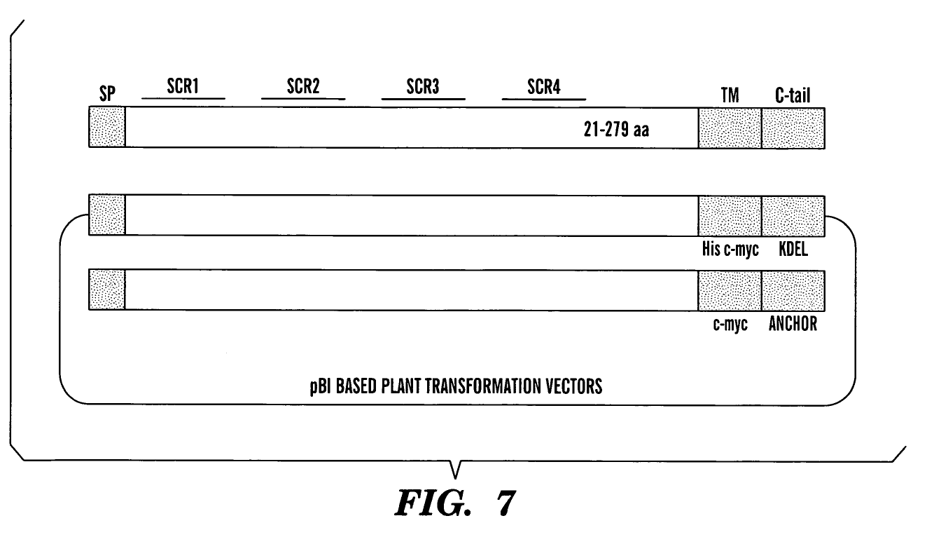

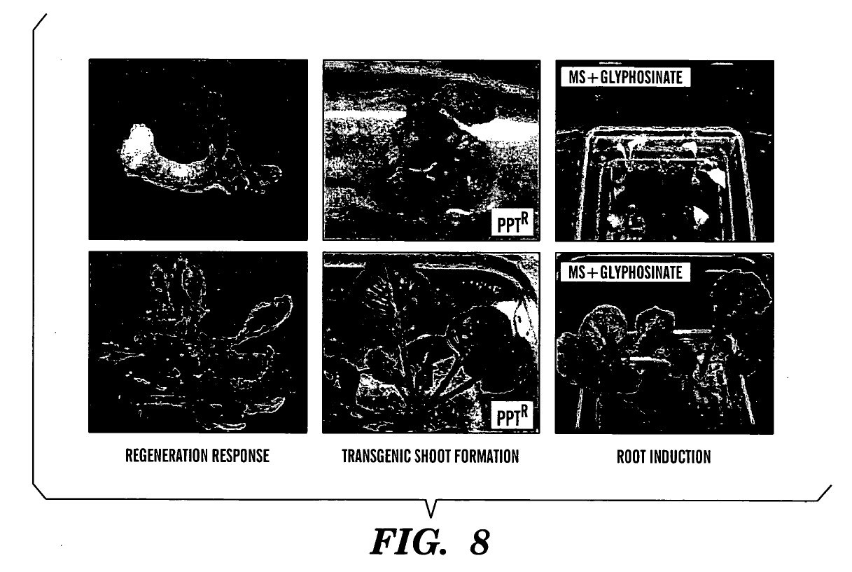

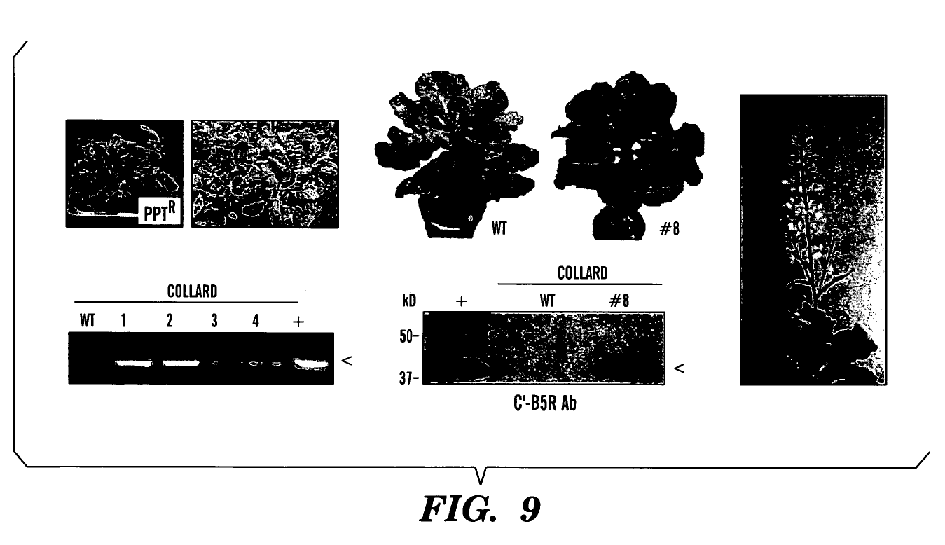

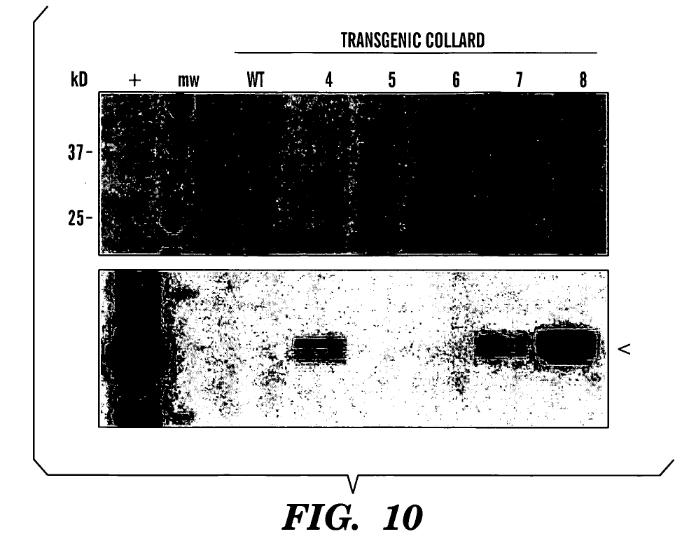

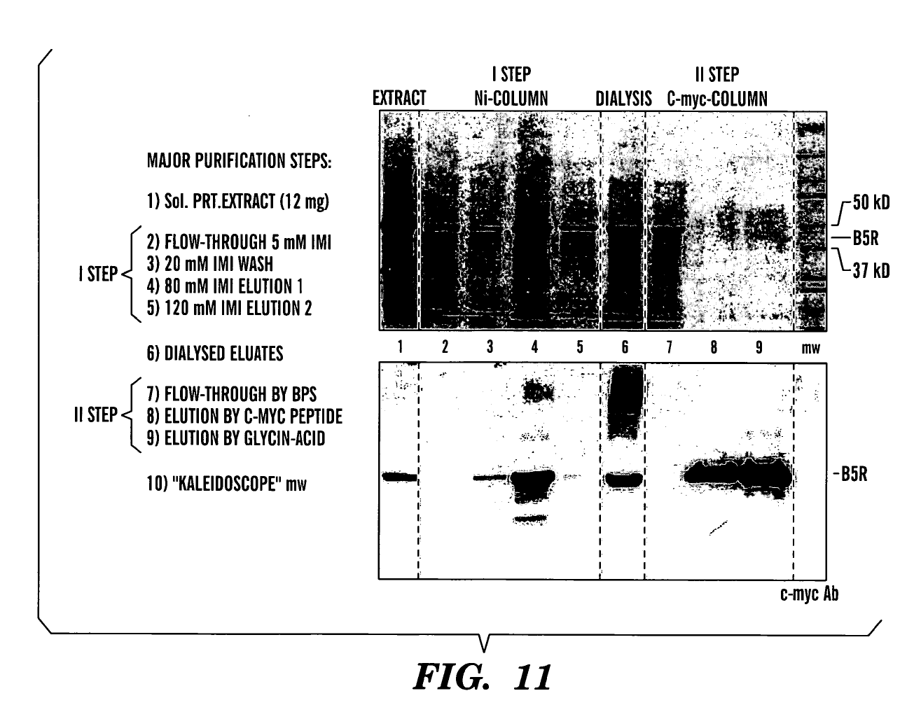

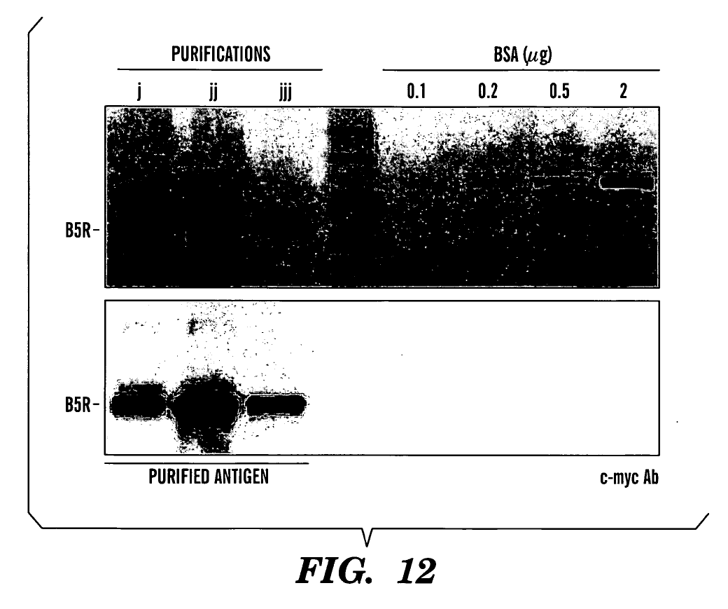

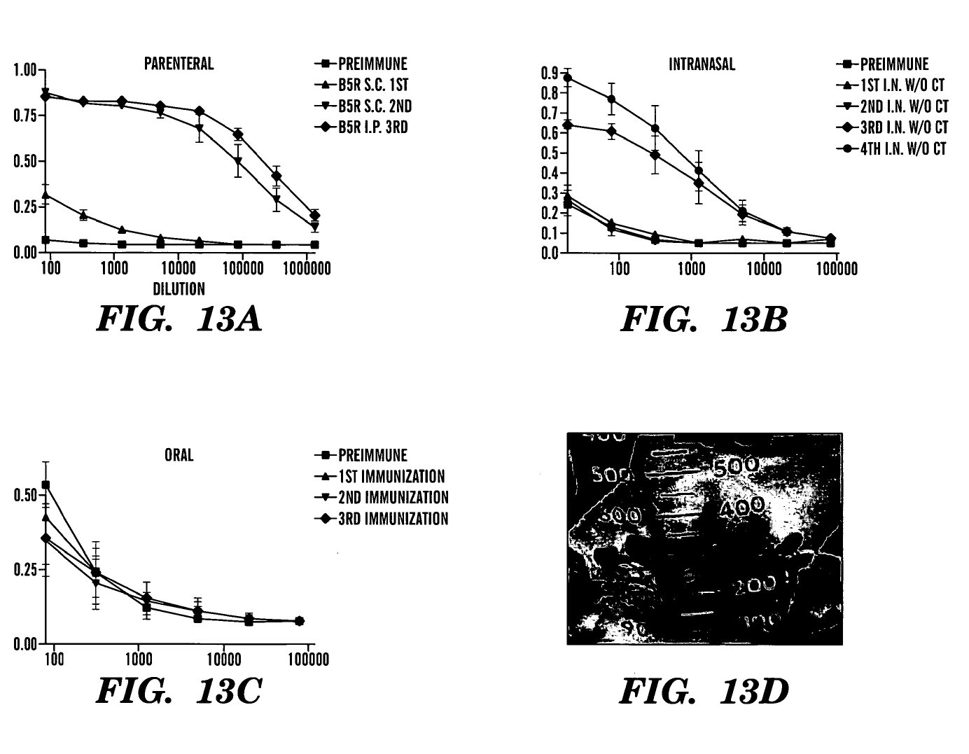

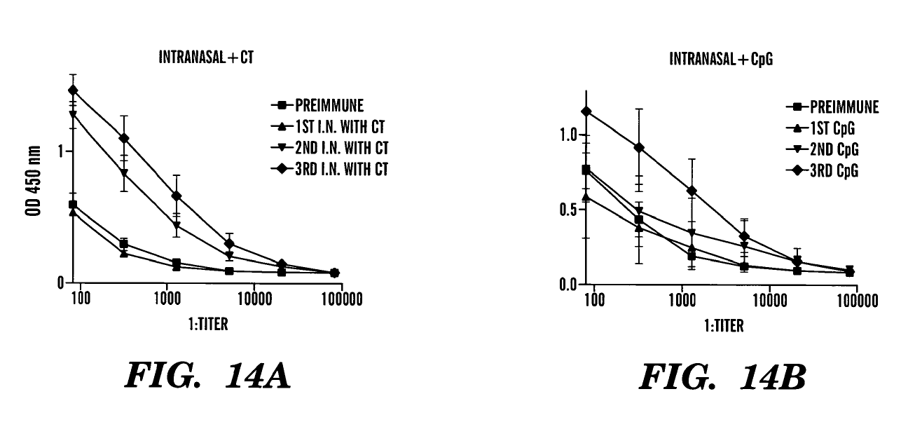

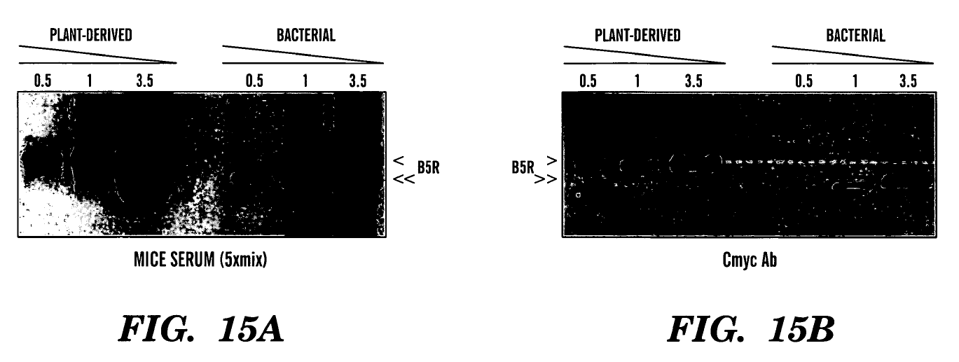

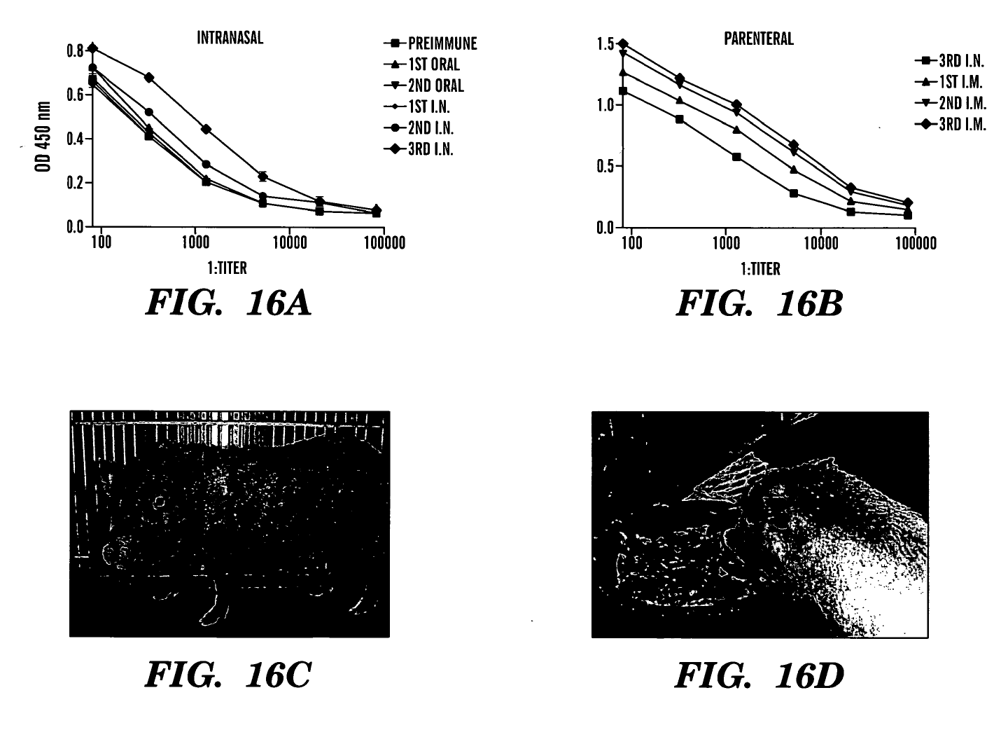

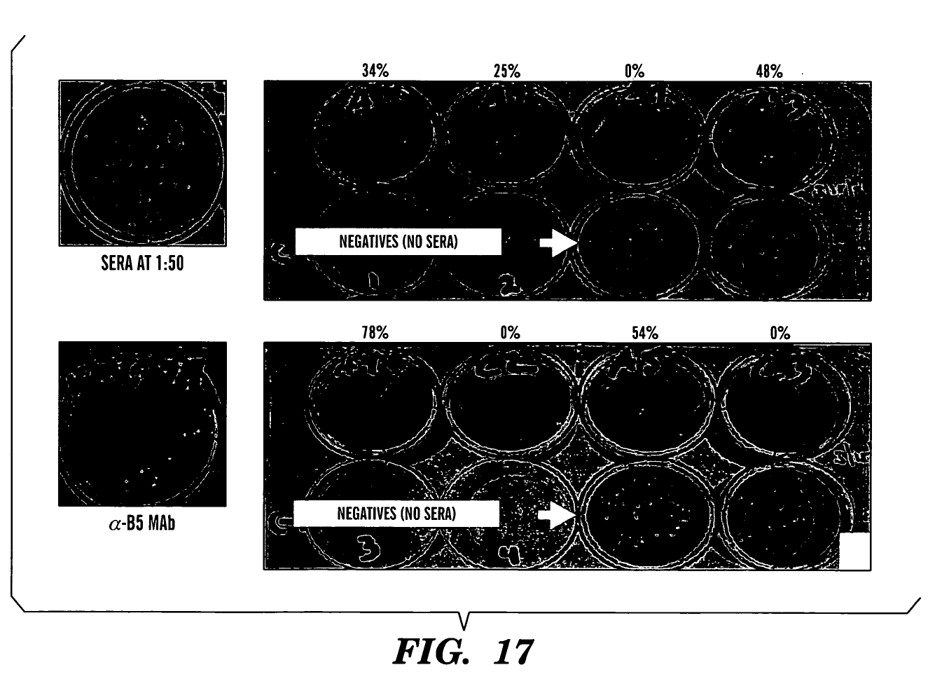

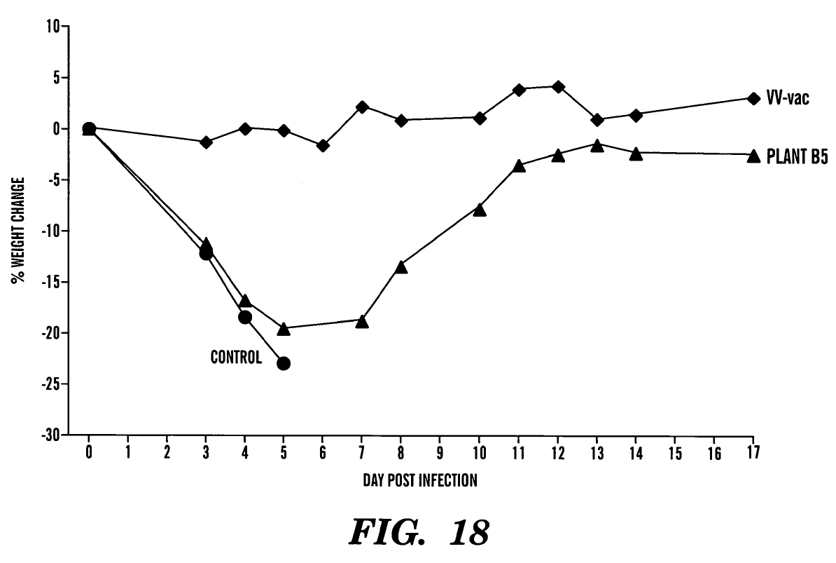

To overcome these problems and develop plant expression system that is capable to produce large amounts of viral antigens, the inventors have discovered a plant-based production of recombinant proteins based on the following considerations. (i) The expression cassettes used must allow the appropriate intracellular localization, folding and post-translational modifications of the recombinant antigens, making them suitable for abundant accumulation in targeted plant cells/tissues as well as being immunologically functional. (ii) The suppression of plant defense mechanisms can be examined using readily available Arabidopsis knockout/mutant plant lines with the capability for sustained high-level expression and accumulation of viral glycoproteins. (iii) For production purposes, the inventors developed crop plants of the same Cruciferae family as the model Arabidopsis plant into an efficient transformation-production system (FIG. 5). The inventors criteria for choosing these crops include: easy scalability for production, overall consumption safety, large production biomass, long-term storage capacity, and ease of processing and delivery. It is expected that results obtained for Arabidopsis are also likely in the related plants. These steps, serve to identify a strategy for the generation of a unique plant-based system for production of large recombinant viral proteins, i.e. components of subunit vaccines, in amounts sufficient for immunization of humans and/or animals preferably via the oral and possibly via other mucosal routes [6, 15, 18, 28].

Here, the inventors have discovered a unique plant-based system for production of large recombinant viral proteins. The inventors have developing an efficient transformation procedure for production of transgenic vegetables from the Cruciferae family (for example, collard and cauliflower). The inventors optimized transformation conditions by altering multiple parameters, such as type of explant, concentration of Agrobacterium inoculum, duration of co-cultivation period, and selection/regeneration schemas.

As a first step, the inventors demonstrated efficient regeneration system for commercial collard and cauliflower cultivars, based on MS medium supplemented with hormones BAP, zeatin, NAA and addition of 20 μM silver nitrate. While only one example of collard cultivar and one example of a cauliflower cultivar were used by the inventors in transformation studies, a survey of several other commercial genotypes showed that all of them exhibit an acceptable regeneration response at these conditions.

The inventors discovered several factors were critical for successful production of transgenic Cruciferae plants (e.g. collard and cauliflower). Both species used in the Examples are highly sensitive to Agrobacterium, so the concentration of the agrobacterial culture used for inoculation was determined to be very important. Dilution of Agrobacterium led to a significantly reduced necrosis in explant tissues. The inventors also discovered a pre-cultivation period was useful for cauliflower (but not necessarily for the production of transgenic collard plants), consistent with previous studies indicating positive results with pre-cultivation for other Cruciferous species [41,49]. For both collard and cauliflower, the inventors were not able to regenerate transgenic shoots when selection was carried out immediately after co-cultivation. Instead, the inventors discovered the combination of a delay period followed by low selection pressure in regeneration medium and increased selection pressure in root induction medium. This schema was demonstrated to be very efficient and allow for fast generation of transgenic cruciferous plants with total elimination of large number of escapes. The inventors demonstrated this approach was efficient for both selection agents used (Km and PPT). Altogether, the inventors demonstrated optimized selection and regeneration procedures for collard and cauliflower yield transgenic plants in a relatively short time of 8-10 weeks from the beginning of the experiment until transfer of transgenic plants to soil. Overall transformation efficiency of collard cv Morris Heading was very high (11%). For cauliflower cv Early Snowball transformation efficiency was 2.4%.

For collard transformation, the inventors used the binary vector carrying expression cassette of smallpox antigen vaccinia virus B5 coat protein driven by a strong CaMV-35S promoter. The 37 kDa B5 extracellular envelope protein is required for formation of infectious virus and for the cell-to-cell and long-range dissemination of the virus in vitro and in vivo. The inventors demonstrate that transgenic collard plants expressing this antigen showed no morphological changes or anomalies compared to control plants. Even the highest expressing line (B5⁺) had the same growth characteristics as control plants. High and stable expression levels of Smallpox antigen were obtained through all stages of collard development in leafy tissues. The expression levels of this antigen in collards were not decreased after several months growth in greenhouse conditions.

In cauliflower, the inventors used a construct containing a SARS-CoV S1 gene driven by the strong Ocs₃Mas promoter. Presently the SARS-CoV S1 protein and its truncated versions are considered the best candidates for generation of a recombinant vaccine against this disease. In a previous study, the inventors demonstrate successful constitutive expression of SARS S1 antigen in Solanaceae plants [18]. As shown herein, the vaccine candidate against SARS was expressed in transgenic cauliflower. The inventors demonstrate that the Ocs₃Mas promoter worked well in florets of cruciferous plants, for example in florets of cauliflower plants.

As demonstrated in the Examples herein, successful transformation of collard plants and production of transgenic collard plants with a viral antigen has been achieved. The inventors have also demonstrated the production of pharmaceutical proteins in cruciferous transgenic vegetables collard and cauliflower. This work opens new possibilities to use cruciferous species, such as collard and cauliflower in transgenic biotechnology.

### REFERENCES

The references cited herein and throughout the application are incorporated herein by reference.
1. M. Hansson, P-A. Nygren, S. Stahl, Design and production of recombinant subunit vaccines. Biotechnol Appl Biochem. 32 (2000) 95-107.
2. T. G. Clark, D. Cassidy-Hanley, Recombinant subunit vaccines: potentials and constraints. Dev Biol. 121 (2005) 153-163.
3. H. Daniell, S. J. Streatfield, K. Wycoff, Medical molecular farming: production of antibodies, biopharmaceuticals and edible vaccines in plants. Trends Plant Sci. 6 (2001) 219-226.
4. JK-C. Ma, P. M. W Drake, P. Christou, The production of recombinant pharmaceuticals proteins in plants. Nature Rev Genet. 4 (2003) 794-805.
5. Y. Gleba, S. Marillonnet, V. Klimyuk, Design of safe and biologically contained transgenic plants: tools and technologies for controlled transgene flow and expression. Biotechnol Genet Eng Rev. 21 (2004) 325-367.
6. H. Koprowski, Vaccines and sera through plant biotechnology. Vaccine. 23 (2005) 1757-1763.
7. G. Giddings, G. Allison, D. Brooks, A. Carter, Transgenic plants as factories for biopharmaceuticals. Nature Biotech. 18 (2005) 1151-1155.
8. D. A. Goldstein, J. A. Thomas, Biopharmaceuticals derived from genetically modified plants. Q J Med. 97 (2004) 705-716.
9. S. J. Streaffield, Plant-based vaccines for animal health. Rev Sci Tech Offlint Epiz. 24 (2005) 189-199.
10. 1K-C. Ma, R. Chikwamba, P. Sparrow, R. Fisher, R. Mahoney, R. M. Twyman, Plant-derived pharmaceuticals the road forward. Trends in Plant Sci. 10 (2005) 580-585.
11. D. T. O'Hagan, R. Rappuoli, Novel approaches to vaccine delivery. Pharm Res. 21 (2004) 1519-1530.
12. S. Mitragotri, Immunization without needles. Nature Rev Immunol. 5 (2005) 905-916.
13. A. Slater, N. W. Scott, M. R. Fowler, Plant biotechnology. The genetic manipulation of plants: Oxford University Press, Oxford, UK, 2003, pp. 1-368.
14. J. M. Dunwell, Transgenic crops: the current and next generations. Methods Mol. Biol. 286 (2005) 377-398.
15. P B. McGarvey, J. Hammond, M. M. Dienelt, D. C. Hooper, iF. Fu, B. Dietzschold, H. Koprowski, F. H. Michaels, Expression of the rabies virus glycoprotein in transgenic tomatoes. Bio/Technology. 13 (1995) 1484-1487.
16. J. S. Sandhu, S. F. Krasnyanski, L L. Domier, 5.5. Korban, M. D. Osadjan, D. E. Buetow, Oral immunization of mice with transgenic tomato fruit expressing respiratory syncytial virus-F protein induces a systemic immune response. Transgenic Res. 9 (2000) 127-135.
17. S. S. Korban. S. F. Krasnyanski, D. E. Buetow, Foods as production and delivery vehicles for human vaccines. 7 of the American College of Nuttition. 21 (2002) 212S-217S.
18. N. Pogrebnyak, M. Golovkin, V. Andrianov, S. Spitsin, Y. Smimov, R. Egolf, H. Koprowski, Severe acute respiratory syndrome (SARS)S protein production in plants: Development of recombinant vaccine. Proc Natl. Acad Sci USA. 102 (2005) 9062-9067
19. H. S. Mason, T. A. Haq, J. D. Clements, C. J. Arntzen, Edible vaccine protects mice against Escherichia coli heat-labile enterotoxin (LT): potatoes expressing a synthetic LT-B gene. Vaccine. 16 (1998) 1336-1343.
20. T. Arakawa, D. K. X. Chong, W. H. R. Langridge, Efficacy of a food plant-based oral cholera toxin B subunit vaccine. Nature Biotechnol. 16 (1998) 292-297.
21. C. O. Tacket, H. S. Mason, G. Losonsky, M. K. Estes, M. M Levine, C. J. Arntzen, Human immune responses to a novel Norwalk virus vaccine delivered in transgenic potatoes. J Infect Dis. 182 (2000) 302-305.
22. 1. Matsumura, N. Itchoda, H. Tsunemitsu, Production of immunogenic VP6 protein of bovine group A rotavirus in transgenic potato plants. Arch Virol. 147 (2002) 1263-1270.
23. J. Yu, W. Langridge, Expression of rotavirus capsid protein VP6 in transgenic potato and its oral immunogenicity in mice. Transgenic Res. 12 (2003) 163-169.
24. S. Biemelt, U. Sonnewald, P. Gaimbacher, L. Willmitzer, M. Muller, Production of human papillomavirus type 16 viral-like particles in transgenic plants. J. Virol. 77 (2003) 9211-9220.
25. L. Erickson, W. J. Yu, J. Brandle, R. Rymenson, Molecular farming of plants and animals for human and veterinary medicine. Kiuwer Academic Publishers, The Netherlands, 2002, pp. 1-400.
26. M. J. Dus Santos, A. Wigdorovitz, K. Trono, R. D. Bios, P. M. Franzone, F. Gil, J. Morenod, C. Carrillo, J. M. Escribano, M. V. Borca, A novel methodology to develop a foot and mouth disease virus (FMDV) peptidebased vaccine in transgenic plants. Vaccine. 20 (2002) 1141-1147.
27. J-L Dong, B-G. Liang, Y-S. Jin, W-J. Zhang, T. Wang, Oral immunization with pBsW6-transgenic alfalfa protects mice against rotavirus infection. Virology. 339 (2005) 153-163.
28. J. Kapusta, A. Modelska, M. Figlerowicz, T. Pniewski, M. Letellier, O. Lisowa, V. Yusibov, H. Koprowski, A. Plucienniczak, and A. B. Legoski, A plant-derived edible vaccine against hepatitis B virus. FASEB J. 13 (1999) 1796-1799.
29. F. B. Bouche, E. Marquet-Blouin, Y. Yanagi, A. Steinmetz, C. P. Muller, Neutralising immunogenicity of a polyepitope antigen expressed in a transgenic food plant: a novel antigen to protect against measles. Vaccine. 21 (2003) 2065-2072.
30. B. Lamphear, J. M. Jilka, L. Kesl, M. Welter, J. A. Howard, S. J. Streatfield, A corn-based delivery system for animal vaccines: an oral transmissible gastroenteritis virus vaccine boosts lactogenic immunity in swine. Vaccine. 22 (2004) 2420-2424.
31. H. Takagi, T. Hiroi, L Yang, Y. Tada, Y. Yuki, K. Takamura, R. Ishimitsu, H. Kawauchi, H. Kiyono, F. Takaiwa, A rice-based edible vaccine expressing multiple T cell epitops induces oral tolerance for inhibition of Th2-mediated IgE responses. Proc Natl Acad Sci USA. 102 (2005) 17525-17530.
32. F. Sala, M. M. Rigano, A. Barbante, B. Basso, A. M. Walmsley, S Castiglione, Vaccine antigen production in transgenic plants: strategies, gene construct and perspectives. Vaccine. 21 (2003) 803-808.
33. G. Van Poppel, D. T. Verhoven, H. Verhagen, R. A. Goldbohm, Brassica vegetables and cancer prevention Epidemiology and mechanisms. Adv Exp Med. Biol. 472 (1999) 159-168.
34. J. W. Lampe, S. Peterson, Brassica, biotransformation and cancer risk: genetic polymorphisms alter the preventive effects of cruciferous vegetables. J. Nutr. 132 (2005) 2991-2994.
35. The Arabidopsis Book, eds. C. R. Somerville and E M. Meyerowitz, American Society of Plant Biologists, Rockville, Md., http://www.bioone.org'bioone/?request=get-toc&issn=1543-8120, pp. 1-1270.
36. E. Passeleue, C. Kerlan, Transformation of cauliflower (Brassica oleracea var. botrytis) by transfer of cauliflower mosaic virus genes through combined co-cultivation with virulent and a virulent strains of Agrobacterium. Plant Science Limerick. 113 (1996) 79-89.
37. L-C. Ding, C-Y. Hu, K-W. Yeh, P-J. Wang, Development of insect-resistant transgenic cauliflower plants expressing the trypsin inhibitor gene from local sweet potato. Plant Cell Rep. 17 (1998) 854-860.
38. P. Bhalla, N. Smith, Agrobacterium tumefaciens-mediated transformation of cauliflower, Brassica oleracea var. botrytis. Mol. Breeding. 4 (1998) 531-541.
39. R. Chakrabarty, N. Viswakarma, S. R. Bhat, P. B. Kind, B. D. Singh, V. L. Chopra, Agrobacteriummediated transfoermation of cauliflower: optimization of protocol and development of Bt-transgenic cauliflower. J. Biosci. 27 (2002) 495-502.
40. J. Puddephat, T. J. Riggs, I. M. Fenning, Transformation of Brassica oleracea L: a critical review. Mol. Breed. 2 (1996) 185-210.
41. S. Dixit, D. K. Srivastava, Genetic transformation in cauliflower—a mini review. J Plant Biol. 27 (2000) 99-104.
42. T. Murashige, F. Skoog, A revised medium for rapid growth and bioassays with tobacco tissue cultures. Physiol Plant. 15 (1962) 473-497.
43. R. B. Horsch, J. Fry, N. Hoffmann, J. Neidermeyer, S. G. Rogers, R. T. Fraley, In: Gelvin S B, Schilperoort R A (eds) Plant Molecular Bioligy Manual. Kluwer Academic Publishers, Dordrecht, Netherlands, 1988, pp.A5/1-A5/9.
44. M. Go!ovkin, A. S, N. Reddy, Functional analysis of pollen-specific calmodulin-binding protein from Arabidopsis. Proc Nath Acad Sci USA. 100 (2003) 10558-63.
45. L. Aldaz-Carroll, J. C. Whitbeck, M. P. de Leon, H. Lou, L. Hirao, S, N. Isaacs, B. Moss, R. J. Eisenberg, G. H. Cohen, Epitope-mapping studies define two major neutralization sites on the vaccinia virus extracellular enveloped virus glycoprotein B5. J of Virol. 79 (2005) 6260-6271.
46. N. V. Chichkova, S. H. Kim, E. S. Titova, M. Kalkunr, V. S. Mrozova, Y. P. Pubstova, N. O. Kalinina, M. E. Taliansky, A. B. Vartapetian, A plant caspase-like protease activated during the hypersensitive response. The Plant Cell. 16 (2004) 157-171.
47. D. Ponder, R. Mittler, E. Lam, Mechanism of cell death and disease resistance induction by transgenic expression of bacteriodopsin. Plant J. 30 (2002) 499-509.
48. N. Hatsugai, M. Kuroyanagi, K. Yamada, T. Meshi, S. Tsuda, M. Kondo, M. Nishimura, I. HaraNishirnura, A plant vacuolar protease, WE, mediates virus-induces hypersensitive cell death. Science. 305 (2004) 855-858.
49. V. Babic, R. S. Datla, G. J. Scoles, W. A. Keller Development of an efficient Agrobacterium-mediated transformation system for Brassica carinata. Plant Cell Rep. 17 (1998) 183-188.

1-41. (canceled)

42. A method for generating a transgenic cauliflower plant, the method comprising;
a. transforming a cauliflower plant cell with a nucleic acid construct comprising a nucleic acid sequence encoding an antigen which is operatively linked to a plant-promoter, wherein the plant-promoter directs expression of the nucleic acid sequence encoding an antigen in cauliflower plant florets; and
b. regeneration of the cauliflower plant cell to produce a transgenic cauliflower plant.

43. The method of claim 42, wherein the promoter is OCS₃ Mas promoter.

44. A transgenic cauliflower plant, wherein the transgenic cauliflower plant expresses a recombinant protein.

45. The transgenic cauliflower plant of claim 44, wherein the recombinant protein is expressed in at least one floret.

46. The transgenic cauliflower plant of claim 44, wherein the recombinant protein is an immunogenic protein.

47. The transgenic cauliflower plant of claim 44, wherein the immunogenic protein is a coat protein or a capsid of a virus.

48. The transgenic cauliflower plant of claim 44, wherein the immunogenic protein is a SARS virus coat protein or a fragment thereof.

49. The transgenic cauliflower plant of claim 48, wherein the fragment of the SARS virus coat protein is an S1-fragment of the SARS-CoV spike protein, or a fragment thereof.

50. A pharmaceutical composition comprising a recombinant protein produced by a transgenic cauliflower plant.

51. A pharmaceutical composition of claim 50, wherein the pharmaceutical composition is administered to a subject for treatment of a disease or disorder, or to induce an immune response in a subject.

52. The method to deliver an immunogenic protein to a subject, wherein the immunogenic protein is expressed by a transgenic cauliflower plant of claim 44 and wherein, wherein the subject ingests an effective amount of a portion of the transgenic cauliflower plant which expresses an immunogenic protein.

53. The method of claim 52, wherein the subject ingests the florets of the transgenic cauliflower plant.

54. A method of storing an immunogenic protein, wherein the immunogenic protein is expressed by a transgenic cauliflower plant of claim 44, wherein the portion of the transgenic cauliflower expressing the immunogenic protein is stored at any temperature between +4° C. to −80° C.

55. A seed of the transgenic cauliflower plant of claim 44.

56. A method for generating a transgenic collard plant, the method comprising;
a. transforming a collard plant cell with a nucleic acid construct comprising a nucleic acid sequence encoding an antigen which is operatively linked to a plant-promoter, wherein the plant-promoter directs expression of the nucleic acid sequence encoding the antigen protein in a transgenic collard plant leaf tissue; and
b. regenerating the collard plant cell to produce a transgenic collard plant.

57. The method of claim 56, wherein the plant promoter is CaMV-35S promoter.

58. A transgenic collard plant, wherein the transgenic collard plant expresses a recombinant protein.

59. The transgenic collard plant of claim 58, wherein the recombinant protein is expressed in at least one transgenic collard leaf tissue.

60. The transgenic collard plant of claim 58, wherein the recombinant protein is an immunogenic protein.

61. The transgenic collard plant of claim 60, wherein the immunogenic protein is a coat protein or a capsid of a virus.

62. The transgenic collard plant of claim 60, wherein the immunogenic protein is a Small Pox virus protein or a fragment thereof.

63. The transgenic collard plant of claim 62, wherein a fragment of the Small Pox virus protein is a fragment of the B5 small pox virus coat protein.

64. The use of the transgenic collard plant of claim 58 for the production of a recombinant protein, wherein the recombinant protein is harvested from the collard green leaf tissues.

65. The use of the transgenic collard plant of claim 64, wherein the harvesting is harvesting the biomass of green leaf tissue.

66. The use of the transgenic collard plant of claim 64, wherein the recombinant protein is purified from the biomass of transgenic collard leaf tissue.

67. A pharmaceutical composition comprising a recombinant protein produced by the method of claim 64.

68. The pharmaceutical composition of claim 67, wherein the recombinant protein is a Small Pox Virus protein or a fragment thereof.

69. The pharmaceutical composition of claim 68, wherein the fragment of the Small Pox Virus protein is a fragment of the B5 small pox virus coat protein.

70. The pharmaceutical composition of claim 67, wherein the recombinant protein is used administered to a subject as a vaccine.

71. The pharmaceutical composition of claim 67, wherein the composition comprises transgenic collard leaf tissue biomass which comprises the recombinant protein.

72. The pharmaceutical composition of claim 67, wherein the recombinant protein is purified from transgenic collard leaf tissue.

73. The pharmaceutical composition of claim 67, wherein the composition further comprises an adjuvant.

74. A method of vaccinating a subject comprising;
a. expressing an immunogenic polypeptide by a transgenic collard plant of claim 58;
b. administering the immunogenic polypeptide to the subject, wherein;
i. the subject ingests an effective amount of a portion of the transgenic collard plant which expresses the immunogenic protein; and/or
ii. the subject is administered an effective amount of the pharmaceutical composition of claim 67, wherein the recombinant protein is an immunogenic protein;
wherein the immunogenic protein induces an immune response in the subject.

75. The method of claim 74, wherein the subject ingests a portion of the transgenic collard plant leaf tissue.

76. The method of claim 74, wherein administration of the pharmaceutical composition is by oral administration.

77. The method of claim 74, wherein the subject is human.

78. A seed of the transgenic collard plant of claim 69.
